SEQUENCE LISTING (93 sequences)

SEQ ID NO: 1 - DNA, 61 bp, Artificial Sequence
  detection probe
ctacattgtcactgcaaatcgacacctattaatgggtctcacctcccaactgcttccccct

SEQ ID NO: 2 - DNA, 66 bp, Artificial Sequence
  detection probe
gagctggatggcctgttggaaatcaaccgcatgacccacaagctgctgagccggtacctgacgctg

SEQ ID NO: 3 - DNA, 65 bp, Artificial Sequence
  detection probe
cagggggtgttgagcgcaggtaggtgtataatagtgaccactgcgtggtggagcagggtaccatg

SEQ ID NO: 4 - DNA, 64 bp, Artificial Sequence
  detection probe
gaagcgtgaggggacagatttgtgaccggcgcggtttttgtcagcttactccggccaaaaaaga

SEQ ID NO: 5 - DNA, 73 bp, Artificial Sequence
  detection probe
ggaccaacacttgtacagcagtgatccattgtatgttccagatgacagggttttggttactgagactcaggtt

SEQ ID NO: 6 - DNA, 73 bp, Artificial Sequence
  detection probe
gaagaagactcagaagcatcttcctcaaggataggtgatagctcacagggagacaacaatttgcaaaaattag

SEQ ID NO: 7 - DNA, 62 bp, Artificial Sequence
  detection probe
cagcgagtctggcgcagagtggagcggccgccggagatgcctgacgcatctgtctgagcatg

SEQ ID NO: 8 - DNA, 65 bp, Artificial Sequence
  detection probe
gcgacatgtgtgggctgcagaccttgcaccccatggatgctgcccagagggaagaacatatcatg

SEQ ID NO: 9 - DNA, 53 bp, Artificial Sequence
  detection probe
gccccgccgctgatccgccaggcgccgccgcccatccgacctgccccaccatg

SEQ ID NO: 10 - DNA, 59 bp, Artificial Sequence
  detection probe
gcgagatccgtgtgtctcccaagatggtggcgctgggctcggggtgactacaggacatg

SEQ ID NO: 11 - DNA, 62 bp, Artificial Sequence
  detection probe
cgatggtatcctgtccgctcgcattggggcgcgtcccccatccgcccccaactgtggtcatg

SEQ ID NO: 12 - DNA, 64 bp, Artificial Sequence
  detection probe
ctctgctgcctcagagaccccaaagtcactgccatatgctctgcaggatccctttgcctgtgta

SEQ ID NO: 13 - DNA, 70 bp, Artificial Sequence
  detection probe
caaagggtgtgccctcaccacccacttgatttttttcattttgccaaaaaggggtatgtctttatcaaag

SEQ ID NO: 14 - DNA, 70 bp, Artificial Sequence
  detection probe
ctcaggcggcattggcgataccaggaattcagcaatatcctttgacaactcaggaatccagtacaggaaa

SEQ ID NO: 15 - DNA, 68 bp, Artificial Sequence
  detection probe
cggacgaccgcattcatggtacaactgcgcttgcgcaagaaggatgtctgcaggggtttctgcccatg

SEQ ID NO: 16 - DNA, 74 bp, Artificial Sequence
  detection probe
gcgtgcacagacgtcaggtaaggatctaaaaatagtgtcacttccctccacggacgtgaggtaaggatctcatg

SEQ ID NO: 17 - DNA, 59 bp, Artificial Sequence
  detection probe
cattcgaggtcggcgcaggaggacaactgcgcttgcgcaggaggcacccagggatcatg

SEQ ID NO: 18 - DNA, 67 bp, Artificial Sequence
  detection probe
gtttggtttgcggcagaagaatctgcatttcgaacaagtgccaggactggtctgaggaacacacgtt

SEQ ID NO: 19 - DNA, 64 bp, Artificial Sequence
  detection probe
cggcaggacgagaaagagtgcctcaagtaccaggtgcccctgcccgacagcatgaagctggagt

SEQ ID NO: 20 - DNA, 56 bp, Artificial Sequence
  detection probe
gacgctgtcaagcaggaggctgccctggctgtgcacacgctgcagagcatggcatg

SEQ ID NO: 21 - DNA, 68 bp, Artificial Sequence
  detection probe
ggtgtggccaagtggggataaagagaagagcaacatctctaatgaccagctccatgctctgctccatg

SEQ ID NO: 22 - DNA, 73 bp, Artificial Sequence
  detection probe
ggtttttgcttggaatcagattcctcgctactccaatatggctttaaccacctcttggtgtctcagctaagaa

SEQ ID NO: 23 - DNA, 61 bp, Artificial Sequence
  detection probe
ggcagatcagctgaagcaggacctgcaggaagcacgcgaggcggagcgaagagccaagcag

SEQ ID NO: 24 - DNA, 65 bp, Artificial Sequence
  detection probe
gctagtcctcagagacactgctgcgagggtagtgggcagtgggattagcctcccgcagagccatg

SEQ ID NO: 25 - DNA, 64 bp, Artificial Sequence
  detection probe
gatgcacagttgctctgggagtcttcagaatagaaactacccatctcaagaggagctcattaag

SEQ ID NO: 26 - DNA, 73 bp, Artificial Sequence
  detection probe
caggaggttgtttgctgtactctcccttgtacagttagctgtctctagtgcctgaatgcactaattgtccttt

SEQ ID NO: 27 - DNA, 58 bp, Artificial Sequence
  detection probe
ggcggggtcgccgtacagcgccgggagggacgcgtatctccaaagcccaatcagagtc

SEQ ID NO: 28 - DNA, 73 bp, Artificial Sequence
  detection probe
ggagttctgggaaatcgttcattcatttacagatgaacagaaaagactcttcttgcagtttacaacgggcaca

SEQ ID NO: 29 - DNA, 62 bp, Artificial Sequence
  detection probe
gggccgcgagtgatgataacctaagaggccggcgcgggcgggcgtgagcggcggaggacatg

SEQ ID NO: 30 - DNA, 70 bp, Artificial Sequence
  detection probe
tgcagacagaaattgaggagcaagatgacattcttgaccggctgacaaccaaagtggacaagttagatgt

SEQ ID NO: 31 - DNA, 65 bp, Artificial Sequence
  detection probe
gcgtcttcgctgcaacgaaatcttccctgcggacattctgctgctctcctccagtgacccccatg

SEQ ID NO: 32 - DNA, 52 bp, Artificial Sequence
  detection probe
tcaggagccctgtaatggccggactgcccttcacctcgcagtggacctgcaa

SEQ ID NO: 33 - DNA, 56 bp, Artificial Sequence
  detection probe
gacccgcgcttccttatccggaaaacgaggccgggaaagggagcgccggggccatg

SEQ ID NO: 34 - DNA, 62 bp, Artificial Sequence
  detection probe
caccccggtgctggtggctgtggtgtgctgcgcccagaggtagggtcgcgggtgggcccatg

SEQ ID NO: 35 - DNA, 68 bp, Artificial Sequence
  detection probe
cttgccagacggcgcagacatgtcagaacaaagtaaggatctgagcgaccctaactttgcagcccatg

SEQ ID NO: 36 - DNA, 71 bp, Artificial Sequence
  detection probe
ggagcttcgctatgcggctgctttaagattctagggttgtacaggcccacgccagacacgacgtctgcatg

SEQ ID NO: 37 - DNA, 59 bp, Artificial Sequence
  detection probe
cccgcaccgccgctcatgtatgccctggccttcggtgcttgcctgggaggtaaggcatg

SEQ ID NO: 38 - DNA, 72 bp, Artificial Sequence
  detection probe
cgaccgcggagcacgtgcactttacctgcgcacttgcagatctttctccaggagtgagtttaaggtcccatg

SEQ ID NO: 39 - DNA, 55 bp, Artificial Sequence
  detection probe
cccagtcttcgacctgctgatccttggatcctgaatctgtgcaaacagtaacgtg

SEQ ID NO: 40 - DNA, 65 bp, Artificial Sequence
  detection probe
ctgctgaggtgatctggcgcagagcggaggaggtgcttggcgcttctcaggctcctcctctcatg

SEQ ID NO: 41 - DNA, 74 bp, Artificial Sequence
  detection probe
gagtcgaaactacatcagtaactcagcacagtccaatggggctgttgtaaaggagaaacaacccagcagtcatg

SEQ ID NO: 42 - DNA, 64 bp, Artificial Sequence
  detection probe
gctcctccaccaggtgctacaattgtacagtacgcagcacaatcagctgatggcacacagcagt

SEQ ID NO: 43 - DNA, 56 bp, Artificial Sequence
  detection probe
ccagcgccgaggcagcctcgctgcgccccatcccgtcccgccgggcactcggcatg

SEQ ID NO: 44 - DNA, 61 bp, Artificial Sequence
  detection probe
ctcagctgtgtaatccgctggatgcggaccagggcgctccccattcccgtcgggagcccgc

SEQ ID NO: 45 - DNA, 58 bp, Artificial Sequence
  detection probe
cattgtcactgcaaatcgacacctattaatgggtctcacctcccaactgcttccccct

SEQ ID NO: 46 - DNA, 79 bp, Artificial Sequence
  detection probe
cagaggggaagaggaaagaggaagaagcgctcagatgctccgcggctgtcgtgaaggttaaaaccgaaaataaaaatgg

SEQ ID NO: 47 - DNA, 67 bp, Artificial Sequence
  detection probe
cgttgagcatctagacgtttccttggctcttctggcgccaaaatgtcgttcgtggcaggggttattc

SEQ ID NO: 48 - DNA, 52 bp, Artificial Sequence
  detection probe
gccacactgccctgagcccaaatgggggagtgagaggccatagctgtctggc

SEQ ID NO: 49 - DNA, 56 bp, Artificial Sequence
  detection probe
gctcccccaaaacgacacgcgcttggaccccgaagcctatggcgccccgtgccatg

SEQ ID NO: 50 - DNA, 58 bp, Artificial Sequence
  detection probe
gcgcggacctagagatcccagaagccacagcgcagcggcccggcccgccactatttcc

SEQ ID NO: 51 - DNA, 67 bp, Artificial Sequence
  detection probe
gcgaccttgttcttcctttccttccgagagctcgagcagagaggactgtgatgagacaggataacag

SEQ ID NO: 52 - DNA, 57 bp, Artificial Sequence
  detection probe
ctgcgacagctcctggaagccggcgcggatcccaacggagtcaaccgtttcgggagg

SEQ ID NO: 53 - DNA, 58 bp, Artificial Sequence
  detection probe
gcgaagactacggaggtcgactcgggagcgcgcacgcagctccgccccgcgtccgacc

SEQ ID NO: 54 - DNA, 76 bp, Artificial Sequence
  detection probe
gggatgaagctgaaatggaatatctgaagatagctcaggacctggagatgtacggtgtgaactactttgcaatccg

SEQ ID NO: 55 - DNA, 55 bp, Artificial Sequence
  detection probe
ggagttggatcggcccctgggacttggcgctcgcgagaggctggagcggccagag

SEQ ID NO: 56 - DNA, 63 bp, Artificial Sequence
  detection probe
ctcctgtcccaggtagggaagaggggctgccgggcgcgctctgcgccccgtttctgcatcatg

SEQ ID NO: 57 - DNA, 58 bp, Artificial Sequence
  detection probe
cttctcctggctgtctctgaagactctgctcagtttggccctggtgggagcttgcatc

SEQ ID NO: 58 - DNA, 58 bp, Artificial Sequence
  detection probe
cggtgatgggcgagcttctcttcaccggggcgcagttgcttctctctgacgtcgcctc

SEQ ID NO: 59 - DNA, 58 bp, Artificial Sequence
  detection probe
cggagaactgggacgaggccgaggtaggcgcggaggaggcaggcgtcgaagagtacgg

SEQ ID NO: 60 - DNA, 68 bp, Artificial Sequence
  detection probe
gagccagagagaggctctgagaagaagaccagcggccccctttctcctcccacagggcctcctgcatg

SEQ ID NO: 61 - DNA, 65 bp, Artificial Sequence
  detection probe
cggacacgcctctttgcccgggcagaggcatgtacagcgcatgcccacaacggcggaggcccatg

SEQ ID NO: 62 - DNA, 64 bp, Artificial Sequence; identical to SEQ ID NO: 4
  detection probe

SEQ ID NO: 63 - DNA, 70 bp, Artificial Sequence
  detection probe
cgtgagttttagacaagctagcttttgtgttgtcttggcggccatatttgtaagaagggtgagaagtatg

SEQ ID NO: 64 - DNA, 52 bp, Artificial Sequence
  detection probe
ctccgcctccagcgcatgtcattagcatctcattagctgtccgctcgggctc

SEQ ID NO: 65 - DNA, 59 bp, Artificial Sequence
  detection probe
cagtccctgcacccaggtttccattgcgcggctctcctcagctccttcccgccgccatg

SEQ ID NO: 66 - DNA, 58 bp, Artificial Sequence
  detection probe
cccttacctggaatctggaatcagcctcttctctgatgaccctgaatctgatccttct

SEQ ID NO: 67 - DNA, 55 bp, Artificial Sequence
  detection probe
cgcgaggatctggagcgaactgctgcgcctcggtgggccgctcccttccctccct

SEQ ID NO: 68 - DNA, 55 bp, Artificial Sequence
  detection probe
ccgctccagataagagtgtgcggaaagcgcggcggggctgagacgcgaccaggac

SEQ ID NO: 69 - DNA, 64 bp, Artificial Sequence
  detection probe
cgcctgaacagccctgtcaaagttgctcgaaagcggaagagaatggtgactggaaatggctctc

SEQ ID NO: 70 - DNA, 59 bp, Artificial Sequence
  detection probe
cgcccgccgtgtacaactaccccgagggcgccgcctacgagttcaacgccgcggccatg

SEQ ID NO: 71 - DNA, 56 bp, Artificial Sequence
  detection probe
gggcttccccgcagcccctgcgcgctcctagagctcgggccgtggctcgtcgcatg

SEQ ID NO: 72 - DNA, 52 bp, Artificial Sequence
  detection probe
tgtgtcaccatgtgggtcccggttgtcttcctcaccctgtccgtgacgtgga

SEQ ID NO: 73 - DNA, 65 bp, Artificial Sequence
  detection probe
ccaagctggtcagcttctacctggagacctacggcgccgagctcaccgctaacgtgctgcgcatg

SEQ ID NO: 74 - DNA, 56 bp, Artificial Sequence
  detection probe
cgcgggtctgggtttccacgcgcgtcaggtcatcaccccggagcccagtgggcatg

SEQ ID NO: 75 - DNA, 70 bp, Artificial Sequence
  detection probe
ggccatggaatctgctgaacaaaaggaacaaggtttatcaagggatgtcacaaccgtgtggaagttgcgt

SEQ ID NO: 76 - DNA, 55 bp, Artificial Sequence
  detection probe
gcaggttcttggtgaccctccggattcggcgcgcgtgcggcccgccgcgagtgag

SEQ ID NO: 77 - DNA, 61 bp, Artificial Sequence
  detection probe
ccccttggtttccgtggcaacggaaaagcgcgggaattacagataaattaaaactgcgact

SEQ ID NO: 78 - DNA, 52 bp, Artificial Sequence
  detection probe
gaaagtcctgtggagcctgcagagccttgtcgttacagctgccccagggagg

SEQ ID NO: 79 - DNA, 52 bp, Artificial Sequence
  detection probe
cgaagagcggccggcgccgtgactcagcactggggcggagcggggcgggacc

SEQ ID NO: 80 - DNA, 65 bp, Artificial Sequence; identical to SEQ ID NO: 40
  detection probe

SEQ ID NO: 81 - DNA, 70 bp, Artificial Sequence
  detection probe
tctcaacagcagtgatgatgggactcagggttgtatgggattgccatgtgtggtgatgtaacaaggtgag

SEQ ID NO: 82 - DNA, 67 bp, Artificial Sequence
  detection probe
cacttacgacgccttcgggacaagttgctgtcagttgctgagcaccgagagatcttccccaacttac

SEQ ID NO: 83 - DNA, 25 bp, Artificial Sequence
  amplification primer
cgcggtcgtagaggtaggttggcgc

SEQ ID NO: 84 - DNA, 30 bp, Artificial Sequence
  amplification primer
gacacaactaaccttacccgctccatcgcg

SEQ ID NO: 85 - DNA, 34 bp, Artificial Sequence
  amplification primer
gtatgtttgtgtggttgtagaggtaggttggtgt

SEQ ID NO: 86 - DNA, 34 bp, Artificial Sequence
  amplification primer
caccaacacaactaaccttacccactccatcaca

SEQ ID NO: 87 - DNA, 30 bp, Artificial Sequence
  amplification primer
gaaggtgcgatagtttttggaagtcggcgc

SEQ ID NO: 88 - DNA, 30 bp, Artificial Sequence
  amplification primer
gacgatctaaattccaaccccgatccgccg

SEQ ID NO: 89 - DNA, 35 bp, Artificial Sequence
  amplification primer
gtggagaaggtgtgatagtttttggaagttggtgt

SEQ ID NO: 90 - DNA, 34 bp, Artificial Sequence
  amplification primer
catcaacaatctaaattccaaccccaatccacca

SEQ ID NO: 91 - DNA, 25 bp, Artificial Sequence
  amplification primer
taggttttttaggaaggagagagtg

SEQ ID NO: 92 - DNA, 24 bp, Artificial Sequence
  amplification primer
cctaaaaccccaactacctaaatc

SEQ ID NO: 93 - DNA, 25 bp, Artificial Sequence
  amplification primer
aggagaataagggtatgtttagtgg

(19) United States
(12) Patent Application Publication
(10) Pub. No.: US 2007/0092883 A1
(43) Pub. Date: Apr. 26, 2007

(54) Methylation specific multiplex ligation-dependent probe amplification (MS-MLPA)

(75) Inventors: Johannes Petrus Schouten, Amsterdam (NL); Anders O.H. Nygren, Amsterdam (NL); Abdellatif Errami, Amsterdam (NL)

Correspondence Address: BROWDY AND NEIMARK, P.L.L.C.;624 NINTH STREET, NW, SUITE 300, WASHINGTON, DC, 20001-5303, US

(73) Assignee: DE LUWE HOEK OCTROOIEN B.V., AMSTERDAM (NL)

(21) Appl. No.: 11/258,380

(22) Filed: Oct. 26, 2005

(51) Int. Cl. C12Q 1/68; C12P 19/34

(52) U.S. Cl. 435/6; 435/91.2

(57) ABSTRACT

An improved multiplex ligation-dependent amplification method is disclosed for detecting the presence of specific methylated sites in a single stranded target nucleic acid, while simultaneously, the quantification of the target nucleic acid sequence can be performed, using a plurality of probe sets of at least two probes, each of which includes a target specific region and non-complementary region containing a primer binding site. At least one of the probes further includes the sequence of one of the strands of a double stranded recognition site of a methylation sensitive restriction enzyme. The probes belonging to the same set are ligated together when hybridised to the target nucleic acid sequence, the hybrid is subjected to digestion by the methylation sensitive restriction enzyme, resulting in non-methylated recognition sites being cleaved. The probes of the uncleaved (methylated) hybrid are subsequently amplified by a suitable primer set.

### BACKGROUND OF THE INVENTION

1. Field of the Invention

The invention relates to a method for detecting the presence of a methylated site at a specific location on a single stranded target sequence, to nucleic acid probes for use in the method and to a kit for performing the method.

2. Description of the Related Art

Copy number changes and CpG methylation of various genes are hallmarks of tumor development but are not yet widely used in diagnostic settings. The recently developed MLPA method has increased the possibilities for multiplex detection of gene copy number aberrations in a routine laboratory. Here we describe a novel robust method: the Methylation-Specific Multiplex Ligation-dependent Probe Amplification (MS-MLPA) which can detect changes in both CpG methylation as well as copy number of up to 40 chromosomal sequences in a simple reaction. In MS-MLPA, ligation of MLPA probe oligonucleotides is combined with digestion of the genomic DNA-probe hybrid complexes with methylation-sensitive endonucleases. Digestion of the genomic DNA-probe complex, rather than double stranded genomic DNA, allowed the use of DNA derived from formalin treated paraffin-embedded tissue samples, enabling retrospective studies. To validate this novel method, we used MS-MLPA to detect aberrant methylation in DNA samples of patients with Prader-Willy syndrome (PWS), Angelman syndrome (AS) or acute myeloid leukemia (AML).

In recent years, the identification of gene specific markers for cancer diagnosis has received much attention. Although the attention is primarily focused on MRNA and protein levels in tumor cells, the variation in expression level of many genes could be caused by changes in copy number and/or methylation status of these genes or their regulators. In neuroblastoma, for example, certain genomic imbalances such as gain of 2p24 and 17q and loss of heterozygosity at 1p36 have been associated with a more aggressive phenotype (Schwab, M., Westermann, F., Hero, B. and Berthold, F. (2003) Neuroblastoma: biology and molecular and chromosomal pathology. Lancet Oncol., 4, 472-480; Westermann, F. and Schwab, M. (2002) Genetic parameters of neuroblastomas. Cancer Lett., 184, 127-147).

A recent study describes the use of micro array chip technology for DNA based clinical diagnostics in B cell chronic lymphocytic leukemia (B-CLL) (Schwaenen, C., Nessling, M., Wessendorf, S., Salvi, T., Wrobel, G., Radlwimmer, B., Kestler, H. A., Haslinger, C., Stilgenbauer, S., Dohner, H. et al. (2004) Automated array-based genomic profiling in chronic lymphocytic leukemia: development of a clinical tool and discovery of recurrent genomic alterations. Proc.Natl.Acad.Sci.U.S.A , 101, 1039-1044).

In CLL, trisomy of chromosomes 12 and 19 and loss of the 13q14 region, the p53, ATM and PTEN genes provide important markers for tumor diagnosis (Westermann, F. and Schwab, M., supra).

In addition to genomic imbalances, epigenetic alterations might serve as an important prognostic marker. In this regard it is of note that recent studies imply that hypermethylation of the p16 gene in ovarian cancer and myeloma is associated with poorer prognosis (Galm, O., Wilop, S., Reichelt, J., Jost, E., Gehbauer, G., Herman, J. G. and Osieka, R. (2004) DNA methylation changes in multiple myeloma. Leukemia, 18, 1687-1692; Katsaros, D., Cho, W., Singal, R., Fracchioli, S., Rigault De La Longrais I A, Arisio, R., Massobrio, M., Smith, M., Zheng, W., Glass, J. et al. (2004) Methylation of tumor suppressor gene p16 and prognosis of epithelial ovarian cancer. Gynecol.Oncol., 94, 685-692).

Alterations of DNA methylation patterns have been recognized as a common change in human cancers. Aberrant methylation of normally unmethylated CpG-rich areas, also known as CpG-islands, which are located in or near the promoter region of many genes, have been associated with transcriptional inactivation of important tumor suppressor genes, DNA repair genes, and metastasis inhibitor genes (Esteller, M. and Herman, J. G. (2002) Cancer as an epigenetic disease: DNA methylation and chromatin alterations in human tumours. J.Pathol., 196, 1-7, and Esteller, M. (2003) Relevance of DNA methylation in the management of cancer. Lancet Oncol., 4, 351-358). Therefore, detection of aberrant promoter methylation of cancer-related genes may be essential for diagnosis, prognosis and/or detection of metastatic potential of tumors. As the number of genes known to be hypermethylated in cancer is large and increasing, sensitive and robust multiplex methods for the detection of aberrant methylation of promoter regions are therefore desirable. In addition, the amount of DNA available for large-scale studies is often limited and of poor quality since this DNA is isolated from formalin treated, paraffin-embedded tissues that have been stored at room temperature for years.

Most current approaches for the detection of methylation are based on the conversion of unmethylated cytosine residues into uracil after sodium bisulphite treatment (Frommer, M., McDonald, L. E., Millar, D. S., Collis, C. M., Watt, F., Grigg, G. W., Molloy, P. L. and Paul, C. L. (1992) A genomic sequencing protocol that yields a positive display of 5-methylcytosine residues in individual DNA strands. Proc.Natl.Acad.Sci.U.S.A, 89, 1827-1831), which are converted to thymidine during subsequent PCR. Thus, after bisulphite treatment, alleles that were originally methylated have different DNA sequences as compared to their corresponding unmethylated alleles. These differences can be exploited by several techniques such as, methylation-specific PCR (MSP), restriction digestion (COBRA), Methylight, direct sequencing, denaturing high performance liquid chromatography (DHPLC), nucleotide extension assays (MS-SnuPE), methylation-specific oligonucleotide (MSO) microarray, or HeavyMethyl (Frommer, M. et al., supra; Cottrell, S. E., Distler, J., Goodman, N. S., Mooney, S. H., Kluth, A., Olek, A., Schwope, I., Tetzner, R., Ziebarth, H. and Berlin, K. (2004) A real-time PCR assay for DNA-methylation using methylation-specific blockers. Nucleic Acids Res., 32, e10; Deng, D., Deng, G., Smith, M. F., Zhou, J., Xin, H., Powell, S. M. and Lu, Y. (2002) Simultaneous detection of CpG methylation and single nucleotide polymorphism by denaturing high performance liquid chromatography. Nucleic Acids Res., 30, E13; Eads, C. A., Danenberg, K. D., Kawakami, K., Saltz, L. B., Blake, C., Shibata, D., Danenberg, P. V. and Laird, P. W. (2000) MethyLight: a high-throughput assay to measure DNA methylation. Nucleic Acids Res., 28, E32; Gitan, R. S., Shi, H., Chen, C. M., Yan, P. S. and Huang, T. H. (2002) Methylation-specific oligonucleotide microarray: a new potential for high-throughput methylation analysis. Genome Res., 12, 158-164; Gonzalgo, M. L. and Jones, P. A. (1997) Rapid quantitation of methylation differences at specific sites using methylation-sensitive single nucleotide primer extension (Ms-SNuPE). Nucleic Acids Res., 25, 2529-2531; Herman, J. G., Graff, J. R., Myohanen, S., Nelkin, B. D. and Baylin, S. B. (1996) Methylation-specific PCR: a novel PCR assay for methylation status of CpG islands. Proc.Natl.Acad.Sci.U.S.A, 93, 9821-9826; Xiong, Z. and Laird, P. W. (1997) COBRA: a sensitive and quantitative DNA methylation assay. Nucleic Acids Res., 25, 2532-2534). However, most of these methods are labor intensive and/or allow the study of the methylation status of only one gene at a time. In addition, most of these techniques are not suitable to study large numbers of paraffin-embedded tissue samples.

The recently developed Multiplex Ligation-dependent Probe Amplification (MLPA) technique (U.S. Pat. No. 6,955,901; both incorporated herein by reference) has been accepted as a simple and reliable method for multiplex detection of copy number changes of genomic DNA sequences using DNA samples derived from blood (Gille, J. J., Hogervorst, F. B., Pals, G., Wijnen, J. T., van Schooten, R. J., Dommering, C. J., Meijer, G. A., Craanen, M. E., Nederlof, P. M., de Jong, D. et al. (2002) Genomic deletions of MSH2 and MLH1 in colorectal cancer families detected by a novel mutation detection approach. Br.J.Cancer, 87, 892-897; Hogervorst, F. B., Nederlof, P. M., Gille, J. J., McElgunn, C. J., Grippeling, M., Pruntel, R., Regnerus, R., van Welsem, T., van Spaendonk, R., Menko, F. H. et al. (2003) Large genomic deletions and duplications in the BRCA1 gene identified by a novel quantitative method. Cancer Res., 63, 1449-1453; Kluwe, L., Nygren, A. O., Errami, A., Heinrich, B., Matthies, C., Tatagiba, M. and Mautner, V. (2005) Screening for large mutations of the NF2 gene. Genes Chromosomes.Cancer, 42, 384-391; Meuller, J., Kanter-Smoler, G., Nygren, A. O., Errami, A., Gronberg, H., Holmberg, E., Bjork, J., Wahlstrom, J. and Nordling, M. (2004) Identification of genomic deletions of the APC gene in familial adenomatous polyposis by two independent quantitative techniques. Genet.Test., 8, 248-256; Slater, H., Bruno, D., Ren, H., La, P., Burgess, T., Hills, L., Nouri, S., Schouten, J. and Choo, K. H. (2004) Improved testing for CMT1A and HNPP using multiplex ligation-dependent probe amplification (MLPA) with rapid DNA preparations: comparison with the interphase FISH method. Hum.Mutat., 24, 164-171), amniotic fluid (Slater, H. R., Bruno, D. L., Ren, H., Pertile, M., Schouten, J. P. and Choo, K. H. (2003) Rapid, high throughput prenatal detection of aneuploidy using a novel quantitative method (MLPA). J.Med.Genet. , 40, 907-912) or tumors (Worsham, M. J., Pals, G., Schouten, J. P., Van Spaendonk, R. M., Concus, A., Carey, T. E. and Benninger, M. S. (2003) Delineating genetic pathways of disease progression in head and neck squamous cell carcinoma. Arch.Otolaryngol.Head Neck Surg., 129, 702-708).

With regard to background of the MLPA technique, detection of specific nucleic acids in a sample has found many applications. One of these applications is the detection of single nucleotide substitutions in genes. Single nucleotide substitutions are the cause of a significant number of inherited diseases and/or may confer a greater susceptibility to display a certain phenotype such as a disease or an infliction. Detection of nucleic acid sequences derived from a large variety of viruses, parasites and other micro-organisms is very important in medicine, the food industry, agriculture and other areas.

The relative quantification of specific nucleic acid sequences has important applications but is more complex and is therefore not routinely performed. One application of the relative quantification of DNA sequences is detection of trisomies such as Down's syndromes which is due to a trisomy of chromosome 21. In cancer cells deletions or amplifications of specific chromosomal areas often occur. Analysis of these can provide important information needed for optimal treatment. One example is amplification of the ERBB2 (Her-Neu) region on human chromosome 17 which defines a specific class of breast tumors requiring treatment different from other breast cancers. Detection of mutations as well as deleted or amplified chromosomal area's can potentially be used to distinguish benign and malignant tumors in small micro-biopts and can provide a fingerprint of a tumor for clonality analysis. Relative quantification of mRNAs is studied for many different reasons among which improved classification and molecular characterisation of tumors. Relative quantification of cytokine mRNAs from in vitro stimulated blood samples can potentially be used to characterise immune responses.

Many methods are known for the detection of specific nucleic acids in a sample. The most sensitive methods currently available rely on exponential amplification of the nucleic acid(s) to be detected e.g. with the use of the Polymerase Chain Reaction (PCR), Ligase Chain Reaction (LCR) or the self-sustained sequence amplification (3SR).

In PCR, nucleic acid oligomers are provided to the sample to enable priming of nucleic acid synthesis on specific sites on the nucleic acid. Subsequently the nucleic acid sequence between the two amplificationprimers is amplified through successive denaturation, hybridisation and nucleic acid polymerisation steps.

Detection of an amplified nucleic acid, a so-called amplicon, can occur in many different ways. Non-limiting examples are size fractionation on a gel followed by visualisation of nucleic acid. Alternatively, specific amplified sequence can be detected using a probe specific for a part of the amplified sequence.

When it is not, or only superficially, known what sequences to look for in a sample, it is advantageous to use a strategy in which a large variety of different sequences can be detected in a single test. When this so-called multiplex amplification is used to determine the relative abundance of various target nucleic acid in the original sample, it is particularly important that the difference in the number of amplified molecules per amplicon is correlated to the difference in the number of target sequences per amplicon in the sample.

To ensure this correlation, a bias in the amplification of sequences not due to a difference in the relative abundance of target nucleic acids in the sample should be avoided as much as possible.

Multiplex nucleic acid amplification methods can be divided in methods in which one amplification primer pair is used for all fragments to be amplified such as RAPD, AFLP and differential display techniques, and methods using a different amplification primer pair for each fragment to be amplified. The currently available amplification techniques using only one primer pair for all fragments to be amplified are typically used to amplify a random subset of the nucleic acid fragments present in a sample. It is not uncommon that more than 50 fragments are amplified in one reaction using these techniques. It has been shown by Vos et al.(1995), Nucleic Acid Research 23, 4407-14. that the Polymerase Chain Reaction as used in AFLP is capable of amplifying large numbers of unrelated fragments with almost equal efficiency provided that these fragments can be amplified with the same set of PCR primers. Relative amounts of amplification products obtained by AFLP can be used to determine relative copy number of specific fragment sequences between samples.

Multiplex methods for the amplification of specific targets typically use a different primer pair for each target sequence to be amplified. The difference in annealing efficiency of different primer pairs result in a strong bias in the amplification of the different amplicons thereby strongly reducing the fidelity of a quantitative multiplex assay. Furthermore the presence of a large number of different primers results in a strongly increased risk of primer dimer formation diminishing the possibility of reproducible amplifying small amounts of target nucleic acids. Amplification of more than 10 specific nucleic acid fragments in one test is therefore not recommended in the art and usually leads to unreliable results.

Nucleic acid detection methods are known from e.g. WO 96/15271 (herein incorporated by reference), providing a method for copying and detecting sequence information of a target nucleic acid present in a sample, into a well characterised DNA template. The method comprises hybridising up to 5 different probe sets of single stranded first and second DNA probes to a target nucleic acid wherein the first and second probe, after hybridisation to the target sequence and subsequently ligation of the probes are used as a template for amplification. The method is suited for the copying of sequence information of RNA or DNA into a DNA template. Said first and/or said second probe further comprises a tag which is essentially non-complementary to said target nucleic acid. The tags are used for the priming of nucleic acid synthesis in the amplification reaction. Such tag can also be used for detection of the resulting amplicon. Thus, said amplification is initiated by binding of a nucleic acid primer specific for said tag. A bias due to difference in primer sequences is avoided by including into the copying action a nucleic acid tag to which amplification primers are directed. Thus, for the analysis of nucleic acid in a sample the sample is provided with one or more DNA probes wherein said probes comprise a first nucleic acid tag and a second nucleic acid tag, optionally denaturing nucleic acid in said sample, incubating said sample to allow hybridisation of complementary nucleic acid in said sample, functionally separating hybridised probes from non-hybridised probes, providing said hybridised probes with at least a first primer, complementary to said first tag, and a second oligomer primer, complementary to said second tag, amplifying at least part of said DNA probes after hybridisation and analysing the amplificate for the presence of amplified products.

Said first and said second probe can only be amplified exponentially by e.g. PCR when the probes are connected. Since connection can essentially only take place when the probes are substantially adjacent to each other, exponential amplification, and thereby detection of the amplicon is only possible if said first and said second probe where hybridised to the target nucleic acid. Non hybridised probes are not exponentially amplified. Removal of non-hybridised and non-ligated probes is therefore not essential, and the reactions can be carried out in the same reaction vessel. Dependent on the temperature, buffer-conditions, ligase-enzyme and oligonucleotides used, the difference in ligation efficiency of oligonucleotides that are perfectly matched to the target nucleic acid and mismatched oligonucleotides can be very large providing increased possibilities to discriminate closely related target sequences.

A similar method is known from WO 97/45559. Both prior art methods however suffer from serious limitations preventing their use for the detection and relative quantification of more than 5 specific nucleic acid target sequences in a single “one-tube” assay in an easy to perform and robust test with unequivocal results using only a small amount of a nucleic acid sample.

The above identified prior art methods were derived from the Ligase Chain Reaction (LCR ; Barany F., Proc.Natl.Acad.Sci.USA, 88:189-93 (1991). In fact, these previous art methods are designed to use two consecutive amplification reactions, starting with several cycles of LCR. In LCR very short hybridisation reactions and therefore high probe concentrations are used. The ligation and amplification reactions are performed in the same reaction vessel, i.e. without sample immobilisation and without removal of non-ligated probe molecules and buffer constituents. All probe oligonucleotides used in the ligation reaction remain therefore present during the amplification reaction. One of the tags used for amplification which is present at the 3′ end of one of the two probe oligonucleotides is however complementary to one of the PCR primers and will therefore provide a template for primer elongation during the PCR reaction. These unligated probe molecules only contain one of the two tags used in the PCR reaction and can therefore not be amplified exponentially but only linearly. During each PCR cycle each picomole of probe will consume one picomole of one of the PCR primers. For each probe pair present, the probe amounts used in the art, 200-500 femtomoles (W097/45559) of each probe, 750-1500 femtomoles (WO96/15271) or 160 fmoles (WO 98/04746) will consume 5-45 picomoles of one of the PCR primers during the 25-30 PCR cycles that are needed when nanogram amounts of human nucleic acids are being analysed. The use of more than 10 probes simultaneously requires, apart from the amounts necessary for exponential amplification of ligated probes, PCR primer amounts in excess of 50 pMoles for the linear amplification of unligated probes (that are not removed, but still present in the reaction mixture) which results in strongly increased amounts of aspecific amplification products. The multiplex methods in the art are therefore limited to the use of a maximum of 5-10 probes per detection reaction. In related previous art methods even higher probe concentrations are used. In WO 98/37230, 5000 femtomoles of each of three probe oligonucleotides is used. In WO 97/19193, 3200 femtomoles probe are used in each assay. These previous art methods are therefore not suitable for multiplex detection of several probes. The high probe amounts used in the previous art reduces the number of probes that can be used simultaneously as well as the sensitivity of the assay.

The above-discussed limitation is solved by the MLPA technique (U.S. Pat. No. 6,955,901) by using probe amounts more than one order of magnitude lower than described in the previous art. Thereto, the first probe of at least one probe set in the mixture was used in an amount of less than 40 femtomoles, and the molar ratio between the said first primer and the first probe amounted at least 200. The use of such substantial low probe amounts and a relatively high molar ratio between the first primer and the first probe also solved the problem of false positive signals due to extension of the probes having the target specific sequence at their 3′ end when hybridized to the target sequence during the PCR reaction, followed by elongation of the complement of the second target specific probe on these extension products as described in detail in W097/45559A and U.S. Pat. No. 6,027,889 (both herein incorporated by reference).

A consequence of this reduced probe amount was that hybridisation reactions were slower. Typically, hybridisation reactions are performed for 16 hrs. This can be reduced by inclusion of certain chemicals and/or proteins in the reactions as is well known in the art. Previous art methods using, or being derived from LCR reactions use typical hybridisation treatments of 1-5 minutes (WO 97/45559).

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

According to the invention, a rapid and easy to apply MLPA based method, Methylation-Specific Multiplex Ligation Dependent Probe Amplification (MS-MLPA) is described, in particular for the detection of changes in methylation status. MS-MLPA also enables simultaneous detection of copy number changes of e.g. up to 40 selected sequences, e.g. in a reaction using comprising only 20 ng of DNA. The general outline of this method is depicted in FIG. 1. Similar to a conventional MLPA assay (U.S. Pat. No. 6,955,901) genomic DNA is first denatured, followed by adding MS-MLPA probes and a hybridization step of preferably about 16 hours. Subsequently, this probe-DNA complex is ligated and digested by methylation-specific enzymes, wherein ligation and digestion can be performed simultaneously. If the site of interest, e.g. a CpG site, is methylated, a normal MLPA product will be detected. If the site is not methylated, the probe-DNA complex will be digested by the methylation-sensitive enzyme and no amplification product is formed. The MS-MLPA method described here extends the MLPA method for multiplex copy number quantification to a method for simultaneous analysis of the copy number, as well as the methylation status of up to 40 sequences in a simple reaction.

Below, the use of the MS-MLPA assay is exemplary demonstrated on DNA samples from Prader-Willy syndrome (PWS) and Angelman syndrome (AS) patients and on DNA samples derived from acute myeloid leukemia (AML) cell lines. Furthermore, it is shown that the MS-MLPA technique according to the invention can also be applied successfully to DNA derived from paraffin-embedded tissues.

### BRIEF DESCRIPTION OF THE DRAWINGS

FIGS. 1A, 1B, 1C and 1D show graphic outlines of different embodiments of the MS-MLPA invention.

FIGS. 1A gives a general outline of the MS-MLPA technique.

FIG. 1B shows a graphic outline of the MS-MLPA invention using different methylation sensitive restriction endonucleases.

FIGS. 1C and 1D show graphic outlines of different embodiments of MS-MLPA for the detection of differential methylation.

FIG. 2 shows the detection of the methylation status of the imprinting center in human chromosome 15 by MS-MLPA.

FIG. 3 shows the detection of aberrant methylation patterns in AML cell lines by MS-MLPA.

FIG. 4 shows verification of MS-MLPA with methylation specific PCR.

FIG. 5 shows verification of MS-MLPA with bisulphite sequencing.

FIG. 6 shows a comparison of MS-MLPA reactions performed on DNA from paraffin embedded and fresh-frozen samples.

### DETAILED DESCRIPTION OF THE INVENTION

Before the invention is discussed in more detail, the following definitions are given. Further definitions will be known to the skilled person and are derivable from text books. Accordingly, conventional techniques of molecular biology and recombinant DNA techniques, which are in the skill of the art, are explained fully in the literature. See, for instance, Sambrook, Fritsch and Maniatis, Molecular Cloning; A Laboratory Manual, Second Edition (1989) and a series, Methods in Enzymology (Academic Press, Inc.).

As used herein, the term “DNA polymorphism” refers to the condition in which two or more different nucleotide sequences can exist at a particular site in the DNA. “SNP” stands for single nucleotide polymorphism.

A complementary nucleic acid is capable of hybridising to another nucleic acid under normal hybridisation conditions. It may comprise mismatches at a small minority of the sites.

As used herein, “oligonucleotide” indicates any short segment of nucleic acid having a length between 10 up to at least 800 nucleotides. Oligonucleotides can be generated in any matter, including chemical synthesis, restriction endonuclease digestion of plasmids or phage DNA, DNA replication, reverse transcription, or a combination thereof. One or more of the nucleotides can be modified e.g. by addition of a methyl group, a biotin or digoxigenin moiety, a fluorescent tag or by using radioactive nucleotides.

As used herein, the term “primer” refers to an oligonucleotide, whether occurring naturally as in a purified restriction digest or produced synthetically, which is capable of acting as a point of initiation of nucleic acid sequence synthesis when placed under conditions in which synthesis of a primer extension product which is complementary to a nucleic acid strand is induced, i.e. in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides.

A primer sequence need not reflect the exact sequence of the template. For example, a non-complementary nucleotide fragment may be attached to the 5′ end of the primer, with the remainder of the primer sequence being substantially complementary to the strand.

As used herein, the terms “target sequence” and “target nucleic acid” refer to a specific nucleic acid sequence to be detected and/or quantified in the sample to be analysed.

As used herein, “amplification” refers to the increase in the number of copies of a particular nucleic acid. Copies of a particular nucleic acid made in vitro in an amplification reaction are called “amplicons” or “amplification products”.

As used herein, “probe” refers to a known sequence of a nucleic acid that is capable of selectively binding to a target nucleic acid. More specifically, “probe” refers to an oligonucleotide designed to be sufficiently complementary to a sequence of one strand of a nucleic acid that is to be probed such that the probe and nucleic acid strand will hybridise under selected stringency conditions. Additionally a “ligated probe” refers to the end product of a ligation reaction between a pair of probes.

As used herein, the term substantially “adjacent” is used in reference to nucleic acid molecules that are in close proximity to one another. The term also refers to a sufficient proximity between two nucleic acid molecules to allow the 5′ end of one nucleic acid that is brought into juxtaposition with the 3′ end of a second nucleic acid so that they may be ligated by a ligase enzyme. Nucleic acid segments are defined to be substantially adjacent when the 3′ end and the 5′ end of two probes, one hybridising to one segment and the other probe to the other segment, are sufficiently near each other to allow connection of the ends of both probes to one another. Thus, two probes are substantially adjacent, when the ends thereof are sufficiently near each other to allow connection of the ends of both probes to one another.

As used herein, the terms “detected” and “detection” are used interchangeably and refer to the discernment of the presence or absence of a target nucleic acid or amplified nucleic acid thereof or amplified probes specific for that target nucleic acid.

As used herein, the term “hot-start” refers to methods used to prevent polymerase activity in amplification reactions until a certain temperature is reached.

As used herein, the terms “restriction endonucleases” and “restriction enzymes” refer to bacterial enzymes each of which cut double-stranded DNA at or near a specific nucleotide sequence, which sequence is referred to as “recognition site”, or “double-stranded recognition site”.

As used herein the term “PCR” refers to the polymerase chain reaction (Mulis et al U.S. Pat. Nos. 4,683,195, 4,683,202 and 4,800,159). The PCR amplification process results in the exponential increase of discrete DNA fragments whose length is defined by the 5′ ends of the oligonucleotide primers.

The term “wild-type” refers to a gene or gene product which has the characteristics of that gene or gene product when isolated from a naturally occurring source. A wild-type gene is that which is most frequently observed in a population and is thus arbitrarily designed the “normal” or “wild-type” form of the gene. In contrast, the term “mutant” refers to a gene or gene-product having at one or more sites a different nucleic acid sequence when compared to the wild-type gene or gene product.

As used herein, “sample” refers to a substance that is being assayed for the presence of one or more nucleic acids of interest.

As used herein, the terms “hybridisation” and “annealing” are used in reference to the pairing of complementary nucleic acids.

Due to its simplicity, the MS-MLPA method described here could serve as a powerful screening tool in tumor classification where often only limited amounts of DNA are available from tissue slices that have been characterized by histological examination. MS-MLPA can be used for the analysis of both methylation as well as copy number changes in DNA derived from blood samples of patients with various disorders such as PWS, AS, Beckwith-Wiedemann syndrome, and FRAXE/FRAXA mediated mental retardation.

It is to be noted that the vast majority of the advantages of the MLPA technique also apply for the present MS-MLPA technique. The principle of MS-MLPA is almost similar to the previously described MLPA , with the main exception that the target sequences detected by MS-MLPA probes contain a restriction site recognized by a methylation sensitive endonuclease, such as HhaI or HpaII, that are sensitive to cytosine methylation of one CpG site in their recognition sequence. Upon digestion with one of these enzymes, a probe amplification product will only be obtained if the CpG site is methylated. The level of methylation was determined by calculating the ratio of the relative peak area of each target probe from the digested sample and from the undigested sample. It is to be understood that any methylated site present in a target nucleotide sequence can be detected by MS-MLPA according to the claimed invention, as long as the site of interest (as part of a double stranded recognition sequence) can be recognized by a methylation sensitive restriction endonuclease, cleaving the nucleic acid when the sequence of interest is unmethylated, and leaving the nucleic acid uncleaved when the sequence is methylated. Further according to the present invention, a plurality of different methylation sensitive restriction enzymes can be used in a single or in separate reactions to detect multiple methylated sites within a single or a pluraliy of target nucleic acid sequences.

In the process of developing MS-MLPA, the genomic DNA was first digested by CpG methylation-sensitive restriction endonucleases and was then denatured and hybridized with the MS-MLPA probes. Unmethylated recognition sites for the restriction endonuclease are digested, preventing the generation of probe amplification products as the two MLPA probe oligonucleotides bind to separate DNA fragments. Although this alternative procedure yielded excellent results, it has several drawbacks compared to the MS-MLPA method according to the claimed invention. First, the location of the restriction endonuclease site was restricted to the vicinity of the ligation site, whereas in MS-MLPA this site can be anywhere in the probe recognition sequence. Second, digestion had to be performed in very small volumes, as the hybridization reaction in MLPA is limited to a maximum sample volume of 5 μl. Third, a separate undigested sample had to be analyzed in order to be able to detect any copy number changes and to quantify the methylation. Fourth, the salt conditions required for restriction endonuclease digestion, prevented complete denaturation of the genomic CpG islands by a simple heating step. Finally, this alternative procedure did not allow analysis of most DNA samples derived from paraffin-embedded tissue, as the DNA could not be completely digested. This is probably caused by partial denaturation of DNA that is extracted from most paraffin-embedded tissues.

The MS-MLPA technique described here is shown to be a robust method and is even suitable for large-scale analysis of DNA extracted from formaldehyde treated paraffin-embedded tissue. In MS-MLPA, the ligation of the probes while hybridized to their target sequence is combined with simultaneous digestion of these complexes with methylation-sensitive restriction endonucleases such as HhaI or HpaII. The use of HhaI appeared to be more effective than HpaII. Conditions of hybridization and ligation are identical or nearly identical to conventional MLPA reactions. In case the ligation and the digestion with the methylation sensitive restriction enzyme are performed simultaneously, which is preferred, the temperature in such a combined ligation/digestion step should be chosen such that both the ligation activity and the methylation sensitive restriction endonuclease activity are efficient. For example, in case HhaI is used as methylation sensitive restriction endonuclease, the temperature is preferably lower than 54° C., e.g. preferably 49° C. It was found that HhaI activity decreases at temperatures above 50° C. Further, to ensure complete digestion of the DNA/MS-MLPA probe complex, the ligation and digestion time should be adjusted accordingly, which is easily performed without any inventive skill by the skilled person. The said time can e.g. be extended from 15 to 30 minutes as compared to a convential MLPA reaction.

In addition, complete digestion is also apparent by the disappearance of at least some MS-MLPA probes in a MS-MLPA reaction whereas incomplete digestion results in general background peak signals of all MS-MLPA probes. In MS-MLPA, genomic DNA is first fully denatured, followed by the formation of a hemimethylated DNA complex with the MS-MLPA probes. Methylation of only the sample DNA strand of this complex is sufficient to inhibit methylation-sensitive digestion, e.g. by HhaI. This is in line with earlier reports, which demonstrated that methylation of one strand is sufficient to block digestion by most methylation-sensitive restriction endonucleases (Bird, A. P. (1978) Use of restriction enzymes to study eukaryotic DNA methylation: II. The symmetry of methylated sites supports semi-conservative copying of the methylation pattern. J. Mol.Biol., 118, 49-60; Gruenbaum, Y., Cedar, H. and Razin, A. (1981) Restriction enzyme digestion of hemimethylated DNA. Nucleic Acids Res., 9, 2509-2515; www.rebase.neb.com).

Like several other restriction endonucleases with a 4-nucleotide recognition site, HhaI also digests single stranded DNA although at a much lower rate. Several MS-MLPA probes used in initial experiments contained an additional HhaI recognition sequence in the stuffer sequence. This stuffer sequence is included in the M13 derived part of the MS-MLPA probes in order to generate size differences between different probe amplification products. Digestion of single stranded DNA by HhaI is presumably dependent on the formation of secondary structures that render the HhaI site temporarily double stranded. Probes to be used in the claimed invention preferably harbor one or more recognition sites for the methylation sensitive restriction enzyme within the hybridizing sequences. If the said recognition sequence is located outside the hybridizing sequences, there is a chance of secondary structure formation, resulting in double stranded, and therewith cleavable, recognition sites for the restriction enzyme.

In addition, a mutation or SNP very close or within the recognition site of the restriction enzyme could influence the digestion and might yield false positive results. Finally, not all methylated sites, in particular CpG's within a promoter region are analyzed by MS-MLPA, but only those CpG's that block digestion of methylation-sensitive endonucleases. When designing the MS-MLPA probes e.g. for detection of methylation in CpG islands, it is highly preferred that one methylation-sensitive restriction site is present within the recognition sequence, because not all CpG sites in a CpG island need to be methylated to silence the transcription of a particular gene (Tischkowitz, M., Ameziane, N., Waisfisz, Q., De Winter, J. P., Harris, R., Taniguchi, T., D'Andrea, A., Hodgson, S. V., Mathew, C. G. and Joenje, H. (2003) Bi-allelic silencing of the Fanconi anaemia gene FANCF in acute myeloid leukaemia. Br.J.Haematol., 123, 469-471; Taniguchi, T., Tischkowitz, M., Ameziane, N., Hodgson, S. V., Mathew, C. G., Joenje, H., Mok, S. C. and D'Andrea, A. D. (2003) Disruption of the Fanconi anemia-BRCA pathway in cisplatin-sensitive ovarian tumors. Nat.Med., 9, 568-574). Thus, if a signal is generated from one MS-MLPA probe but not from a second probe located elsewhere in the same promoter, this indicates that the particular gene is methylated and additional tests should be performed.

The sensitivity of the MS-MLPA probes for the methylation status of sample DNA can e.g. be demonstrated by the use of human sample DNA that is methylated in vitro by HhaI methylase. This results in amplification products for all probes, as the HhaI endonuclease is unable to cut methylated CpG sites. Specificity of MS-MLPA can further be demonstrated by the observation that most MS-MLPA probes that recognize a HhaI site within a CpG island result in the absence of amplification products after HhaI digestion of DNA samples from healthy individuals.

In contrast, the great majority of MS-MLPA probes that recognize a HhaI site outside a CpG island show the presence of an amplification product upon HhaI digestion of the sample DNA-probe hybrids. This is in agreement with the observation that CpG sites within CpG islands are unmethylated whereas the great majority of isolated CpG sites are methylated in human DNA (Laird, P. W. (2003) The power and the promise of DNA methylation markers. Nat.Rev.Cancer, 3, 253-266).

Several aspects contribute to the benefit of MS-MLPA: (i) a large number of genes can be studied using a minimum amount of e.g. only 20 ng sample DNA; (ii) due to its simple procedure, large numbers of samples can be analyzed simultaneously; (iii) MS-MLPA is quantitative and can discriminate between methylation of one, both or none of the alleles; (iv) the optional simultaneous ligation and digestion reaction enables MS-MLPA to be used on paraffin-embedded tissue samples, because DNA degradation and partial DNA denaturation during embedding of the tissues or longtime storage appear not to influence the results. 64Further, although not necessary, it is advantageous to use a low probe amount as practised in the MLPA technique as disclosed in U.S. Pat. No. 6,955,901. Accordingly, a plurality of probe sets can be used for detecting one or more specific nucleic acid sequences, without the above-mentioned drawback that the probes are significantly consumed by amplification of unligated probes. In order to detect a plurality of different target nucleic acid sequences, the first probes from the probe sets, specific for hybridising to the corresponding nucleic acid sequences and containing a tag complementary to one of the amplification primers, are present in the mixture in the above-mentioned amount.

Preferably, the amount of at least the first probe of each probe set in the mixture is less than 40 femtomoles, the molar ratio between the first primer and the first probe being at least 200. The probe sets differ from one another in that at least one of the probes of different probe sets have different target specific regions, therewith implicating that each probe set is specific for a unique target nucleic acid sequence. However, probe sets may only differ in one of the probes, the other probe(s) being identical. Such primer sets can e.g. be used for the determination of a specific point mutation or polymorphism in the sample nucleic acids.

The molar ratio between the first primer and the first probe is preferably at least 500, more preferably at least 1000, and most preferably at least 2000. The higher the said ratio, the more different primer sets for the detection of a corresponding number of different amplicons can be used. However, as indicated above, unspecific amplification reactions as a result of high primer concentrations is to be avoided. Thereto, the primer concentration preferably is below 50 pMoles, more preferably below 20 pMoles in a reaction volume of 10-100 μl.

Preferably, the molar amount of at least the first probe of at least one probe set, preferably of a plurality of probe sets, more preferably of each probe set in the mixture is less than 10 femtomoles, preferably less than 4 femtomoles. By such low probe amounts, reliable amplification of up to 40 different sets of probes can be achieved. In a multiplex assay as described in examples 1-3, 2 femtomoles each of 40 different probe pairs is used in one assay on 5-100 ng amounts of human chromosomal target DNA. During the at least 30 PCR cycles of the amplification reaction 30×2×40=2400 femtomoles of one of the PCR primers is consumed by linear amplification of unligated probes corresponding to 24% of the available 10 picomoles PCR primer.

Preferably, the probes of the same probe set are present in the mixture in substantially equal amounts, although the said amounts can differ from one another, e.g. dependent on the hybridisation characteristics of the target specific regions with the target nucleic acid sequence. However, the amount of second probe may optionally be a factor 1-5 higher than that of the corresponding first probe, without negatively affecting the reaction.

Although it is possible for the first probe of different probe sets to have different tag sequences, implicating that a plurality of different first primers are to be used in the amplification step it is highly preferred that the first tag sequences of the first nucleic acid probes of the different probe sets are identical, so that only one first primer has to be used in the amplification reaction. A bias in the amplification due to a difference in the sequence of different primers used for the amplification can thus be completely avoided, resulting in a substantially uniform amplification for all probe assemblies. According to the invention it is however also possible that a number of first nucleic acid probes comprise the same tag sequence, whereas first probes belonging to another probe set may comprise another first tag sequence.

In a preferred embodiment, the amplification step comprises binding of a second nucleic acid primer, specific to the second tag sequence, to the elongation product of the first primer. By the use of a second primer, the amplification reaction is not linear, but exponential. Said first and said second probe preferably each comprise a different tag. Preferably said amplification of connected probes is performed with the use of the Polymerase Chain Reaction (PCR).

For the same reasons as discussed above, the molar ratio between the second primer and the second probe is preferably at least 200, more preferably at least 500, even more preferably at least 1000 and most preferably at least 2000.

In line with the above, preferably the second tag sequences of the second nucleic acid probes of the different probe sets are identical, so that for amplification of the primer assemblies a limited amount of different primers may be used. In this way, amplification of all possible primer assemblies can be accomplished using a limited number of primer pairs, preferably only one primer pair. As in such a case, all the probes comprise the same first tag and the same second tag, thereby excluding any bias in the amplification of the probes due to sequence differences in the primers.

In order to prevent competition during a PCR reaction between probe and primer binding in case a single second primer is used in the reaction mixture, the molar ratio between the second primer and the total amount of second probes present in the reaction mixture is preferably at least 5, more preferably at least 15 and most preferably at least 25.

However, it is of course possible to use probes that comprise different first tags and/or different second tags. In this case it is preferred that the primers are matches for similar priming efficiencies. However, some bias can be tolerated for non quantitative applications or when the bias is known, it can be taken into account in a quantitative application.

Because of the preferred low amounts of probes present in the reaction mixture, the number of different probe sets in one reaction may exceed the maximum number of probe sets that can be achieved with the multiplex methods known in the art. The reaction mixture preferably comprises at least 10 probe sets, preferably at least 20 and most preferably 30-40 different sets of probes. It is to be understood that it is preferred to use lower probe amounts when the number of different probe sets increases. Using e.g. 10 different probe sets, the amount of each first probe is preferably less than 20-40 femtomoles, whereas when 30-40 different probe sets are used, the amount of each different first probe is preferably in the range of 1-8 femtomoles in the reaction mixture.

As indicated above, the presence of a second, or further additional, distinct methylated target nucleic acid can be detected with the method according to the present invention. To enable this it is preferred that said sample is provided with at least two probe sets, i.e. the target specific regions of at least one of the first, second, or, when present, the third probes of each set differ from one another. In this case at least two different amplicons can be detected. For instance when a first or said second nucleic acid probe of a probe set is capable of hybridising to (methylated) target nucleic acid essentially adjacent to a probe of the second probe set. Successful connecting of probes can then result in an amplicon resulting from the connection of said first and said second probe of the first set and an amplicon resulting from the connection of the first and second of the second set. For enabling detection of each additional target nucleic acid one can similarly provide additional probes. This has applications for the detection and relative quantification of more than one target nucleic acid which need not be in the same chromosomal region.

To allow connection of essentially adjacent probes through ligation, the probes preferably do not leave a gap upon hybridisation with the target sequence. In that case the first and second segments of the target nucleic acids are adjacent. However, it is also possible that between the first and second segments a third segment is located on the target nucleic acid. In that case a third probe may be provided in a probe set complementary to the third segment of said target nucleic acid, whereby hybridisation of the third probe to said third segment allows the connecting of the first, second and third probes. In this embodiment of the invention a gap upon hybridisation of the first and second probes to the target nucleic acid is filled through the hybridisation of the third probe. Upon connecting and amplification, the resulting amplicon will comprise the sequence of the third probe. One may choose to have said interadjacent part to be relatively small thus creating an increased difference in the hybridisation efficiency between said third segment of the target nucleic acid and the third probe that comprises homology with said third segment of said target nucleic acid, but comprises a sequence which diverges from the perfect match in one or more nucleotides. In another embodiment of the invention a gap between first and second probes on said target nucleic acid is filled through extending a 3′ end of a hybridised probe or an additional nucleic acid filling part of an interadjacent part, prior to said connecting.

Preferably at least a portion of the probes, not hybridised in the incubation step are not removed in the course of the method according to the invention and remain in the reaction mixture together with the hybridised probes.

In the method of the present invention, reaction conditions are used that do not require unligated probe removal or buffer exchange.

With “portion” an amount of probes is meant above trace-level that may remain present when the reaction is subjected to a treatment for complete separation of hybridised probes from unhybridised probes. Preferably, said portion is at least 5% from the unhybridised probes, more preferably 10% or more.

In several multiplex methods in the art, such as WO98/04746, immobilisation of sample nucleic acids is required in order to exchange buffer solutions and remove non target bound probe molecules. Hybridised probes can be separated from non-hybridised probes in a-number of different ways. One way is to fix sample nucleic acid to a solid surface and wash away non-hybridised probes. Washing conditions can be chosen such that essentially only hybridised probes remain associated with the solid surface. The hybridised probes can be collected and used as a template for amplification. According to WO98/04746, probe separation was accomplished by addition of a tagged third target specific oligonucleotide.

It is preferred not to remove any of the unhybridised probes from the reaction mixture, i.e. that all unhybridised probes remain in the reaction mixture during the incubation step, the connecting step and the amplifying step. It is however possible to remove a portion of the unhybridised probes from the mixture if desired. The skilled person is aware of suitable methods for such partial removal. By not removing any of the unhybridised probes from the reaction mixture, the method according to the invention, provides the possibility for an essential one-tube assay using more than 5 probes simultaneously and less than 10.000 copies of each target nucleic acid for each assay.

It is very attractive for the method to be carried out as a “one tube” assay; i.e. the contacting step, the connecting step, the digestion step and preferably also the amplification step are carried out in the same reaction vessel, the reaction mixture not being removed from the said vessel during the said steps.

The contacting, incubation and connecting step are usually carried out in a relatively small volume of 3-20 μl, although larger volumes, as well as increase of volume of the reaction mixture in subsequent reaction steps are tolerated. The amplification step is usually performed in a larger volume of 20-150 μl; for this, the optionally smaller volume of the reaction mixture in the connection step is usually completed to the desired volume for the amplification by adding the additional ingredients for the amplification reaction. In particular, in a typical reaction mixture of 3-150 μl, the amount of: sample nucleic acid is 10 - 1000 ng, the first probe of each probe set is 0,5-40 fmol, the second probe of each probe set is 0-40 fmol, each first primer is 5- 20 pmol, each second primer is 0-20 pmol.

In case that probe sets comprise a second probe, the amount of the second probe is 0,5-40 fmol; in case a second primer is used for the amplification reaction, the amount of the said second primer is preferably 5-20 pmol.

Preferably, the reaction mixture comprises, at least during step 2), ligation activity, by which the first, second and optionally the third probes are, once hybridised to the target nucleic acid and arranged adjacently to one another, connected to one another.

In step 3), the double-stranded probe assemblies are subjected to digestion by the methylation sensitive restriction endonuclease. As it is important that the cleaved nucleic acids are not religated to one another, any ligation activity present, at least during the said step 3), is incapable of ligating double stranded nucleic acids. This can be done e.g. by inactivting the ligase activity from step 2) before adding the methylation sensitive restriction endonulease, or, and preferably, a ligase activity is used, capable of ligating single-stranded nucleic acids to one another, such as the probes, hybridized to the target nucleic acid, while being incapable of ligating double-stranded nucleic acids. By using such a ligase activity, known in the art, e.g. as NAD dependent ligases, steps 2) and 3) can be performed simultaneously.

Another limitation of previously described ligation dependent amplification methods is that the ligation reaction was performed at low temperatures not permitting sufficient hybridisation selectivity for use on complex nucleic acid samples or that thermostable ligases were used that cannot easily be inactivated before the start of the amplification reaction. In a preferred embodiment of the current invention said ligation is performed with a thermostable nucleic acid ligase active at temperatures of 50° C. or higher, but capable of being rapidly inactivated above approximately 95° C. Once probes are connected it is preferred that essentially no connecting activity is present during amplification since this is not required and can only introduce ambiguity in the method. Since amplification steps usually require repeated denaturation of template nucleic acid at temperatures above 95° C. it is preferred to remove the connecting activity through said heat incubation. In order to prevent hybridisation of probes to sequences only partially complementary it is preferred to perform the ligation reaction at temperatures of at least 45° C. It is however important, if the ligation step 3) is performed together with the digestion step 2), that the reaction temperature allows the required digestion to take place. The present invention therefore in one aspect provides a method wherein ligation of probes annealed to a target nucleic acid is performed by a thermostable nucleic acid ligation enzyme, i.e. with an activity optimum higher than at least 45° C., under suitable conditions, wherein at least 95% of the ligation activity of the said ligation enzyme is inactivated by incubating said sample for 10 minutes at a temperature of approximately 95° C.

Another important limitation of the prior art is that only synthetic production of oligonucleotides is used. Synthetic produced oligonucleotides are cheap, essentially pure and are available from many suppliers. Synthetic production of long oligonucleotides has however serious limitations. The length of the complementarity region with the target nucleic acid in the probe is preferably long enough to allow annealing at elevated temperatures. Typically the length of the complementarity region is at least 20 nucleotides. The probes also contain a tag which can be of any size, however, typically a tag comprises a nucleic acid with a length of at least 15 nucleotides. A probe comprising a tag therefore typically comprises a length of 35 or more nucleotides. Amplicons of connected first and second probes typically have a length of at least 70 nucleotides. This minimum length is also preferred to discriminate amplicons from primer dimers and other side products that are often formed in PCR reactions in which only very small amounts of starting template are used.

A problem, particularly encountered in multiplex amplifications, is the discrimination of the different amplicons that can result from the amplification. Discrimination can be achieved in a number of different ways. One way is to design the multiplex amplification such that the size of each amplicon that can occur, is different. Size fractionation on for instance a gel and determination of the size of the detected amplicon then allows discrimination of the various amplicons. Alternatively, amplicons can be discriminated between on the basis of the respective sequences present in the amplicon. For instance through hybridising amplicons to specific probes. However, the latter method has the disadvantage that additional steps need to be included to detect and/or discriminate the amplicons. In the examples illustrating the present invention therefore the various amplicons were discriminated on the basis of size.

However, the discrimination of amplicons which differ only slightly in size is difficult. For optimum quantification of peaks in an electropherogram a size difference between different amplicons of at least 3 nucleotides is preferred. On the other hand longer probes, to allow more differences in size of the resulting amplicons, are not very easily synthesised synthetically. For proper discrimination of a plurality of different amplicons, preferably at least 10, more preferably at least 20 and most preferably 30-40 different amplicons on the basis of size and for optimal quantitation of amplicons, at least one of the probes of a number of amplicons is more than 50-60 nucleotides in size.

Oligonucleotides longer than 60 nucleotides however typically suffer from less yield, lower purity and the reliability of the sequence of the probe becomes a problem. Chemically synthesised oligonucleotides are made stepwise in a 3′- 5′ direction. Coupling yield for each nucleotide is usually only 98,5%, resulting in the presence of a large number of different side products. Besides there is a risk on damaging the already synthesised part of the oligonucleotide during each new cycle of chemical polymerisation. A high reliability of the sequence of a probe is particularly important when already one false nucleotide can give false results.

In an attractive embodiment of the invention, this problem is overcome by utilising at least one probe comprising nucleic acid that is generated through enzymatic template directed polymerisation, at least prior to the hybridisation step. In this embodiment, the above-discussed probe amounts and relative primer-to-probe ratios are preferred. Enzymatic template directed polymerisation can be achieved for instance in a cell. It is preferably achieved through the action of a DNA polymerase, RNA polymerase and/or a reverse transcriptase. Such enzymatic template directed polymerisation is capable of generating large stretches of nucleic acid with a high fidelity, thereby enabling the generation of a reliable probe, that is substantially larger than currently reliably possible with the synthetic methods. A probe comprising nucleic acid that is generated through enzymatic template directed polymerisation is in the present invention further referred to as an enzymatic probe.

Using at least one enzymatic probe it is possible to increase the size differences between the various amplicons.

For multiplex analysis of ligation products using the length of the ligation product to identify the specific ligation products, at least one of the two oligonucleotides will have a length of more than 60 nucleotides in most (but not necessarily all) of the probes. Fragments substantially longer than 60 nucleotides are difficult to synthesise chemically in high yield and high quality. We discovered that fragments derived by restriction endonuclease digestion of plasmids, phages or phagemids are a preferred source of one of the two oligonucleotides used in ligatable probe amplification. These fragments typically contain less than one mistake in every 10.000 bp as template directed enzymatic nucleotide polymerisation occurs with high fidelity and is backed in vivo by several repair mechanisms. Alternatively fragments of a sufficient long length and having a sequence tag can be produced by in vitro enzymatic template directed nucleotide polymerisation as described in example 8 of U.S. Pat. No. 6,955,901. The other probe oligonucleotide to be ligated can be smaller and is most easily produced chemically.

Chemically synthesised oligonucleotides are made in a 3′- 5′ direction. As coupling yield for each nucleotide is usually only 98,5%, a considerable number of fragments in unpurified oligonucleotides are shorter than the required oligonucleotide. The oligonucleotide end involved in the ligation reaction should however be constant. For the experiment described in example 1 we therefore chose to use chemically synthesised oligo's of which the 3′-end is joined by ligation to the 5′-end of the long (enzymatic produced) fragment (Type A probe). The 5′-end of DNA fragments produced by restriction enzyme digestion is phosphorylated. The smaller chemically synthesised oligonucleotide (type B probe) does not have to be phosphorylated as only the 3′-end is used for the ligation reaction. In case of SNP analysis, the SNP site should be close to the end, preferably at the end or at the penultimate site of the chemically synthesised oligonucleotide in order to obtain the largest difference in ligation efficiency between matched and mismatched oligonucleotides.

In a preferred embodiment, the long enzymatic produced oligonucleotide is made by an amplification reaction such as PCR with the use of two primers, one of which contains a sequence tag at its 5′end. In another preferred embodiment of the invention the long oligonucleotide is produced by restriction enzyme digestion of a plasmid or phage clone. In a further preferred embodiment, the 5′-end of the long fragment (type A probe) to be ligated should be complementary to the target nucleic acid. Some restriction endonucleases, among which the commercially available Bsm 1 isolated from Bacillus stearothermophilus NUB36 cleave the DNA outside their DNA recognition site and provide a means to produce oligonucleotides that have a 5′ end with perfect complementarity to the target nucleic acid. Other restriction endonucleases such as Sph I and Aat II produce oligonucleotides that have left only one nucleotide of the restriction enzyme recognition site at the 5′ end of the fragment produced and can be used for the production of some type A probes.

Size differences can be generated by increasing the length of the hybridising region of a probe or by introduction of a stuffer region that is not complementary to the target nucleic acid. By varying the size of the stuffer one can easily design probes that comprise the same hybridisation capacity (wherein the length of complementarity region with the target nucleic acid and the CG/AT content are adjusted to each other), while still being able to discriminate the resulting amplicons by size. Another advantage of non-hybridising stuffer sequences is that stuffer sequences with known amplification characteristics can be selected. Certain DNA sequences have a lower amplification efficiency in amplification reactions for instance due to polymerase pause sites such as hairpins. Stuffer sequences provide the possibility to use long amplification products while knowing that a major part of the probe has good amplification characteristics. In SNP/mutation screening the use of a short hybridising region in combination with a non-hybridising stuffer sequence provides the possibility to simultaneously use probes for SNP's or mutations that are close to each other without competition between probes during the hybridisation reaction while still using the advantages of long amplification products. The stuffer can of course also be used to introduce a tag, for instance for later discrimination of probe amplification products on the basis of stuffer sequence. In one aspect of the current invention a series of cloning vectors each containing different stuffer sequences is provided.

In a preferred embodiment of the invention, one of the probe oligonucleotides is generated by digestion of DNA, in particular plasmid, phage or viral DNA with a restriction endonuclease (also referred to as “probe generating restriction enzyme”, to clearly emphasize the difference with the above-discussed methylation sensitive restriction enzyme, athough the probe generating restriction enzyme may very well be methylation sensitive as well.).

In a further preferred embodiment of the invention one of the probe oligonucleotides is obtained by restriction enzyme digestion of single stranded phage DNA that is made partially double-stranded by annealing of short oligonucleotides. The use of single stranded phage or phagemid DNA increases the effective probe concentration during hybridisation and reduces the amount of probe DNA present as well as the possibility of non-specific amplification products formed e.g. by elongation of one of the PCR primers or one of the short probe oligonucleotides at (partially) complementary sequences of the complementary probe oligonucleotide. In a further preferred embodiment the restriction enzyme is capable of cutting at least one strand of the DNA outside the enzyme recognization site sequence on said DNA, resulting in DNA fragments not containing any residues of the restriction enzyme recognition sequence at their ends. Digestion means cleavage of both or only one strand of a double stranded DNA, such as e.g. cleavage by the restriction enzyme BsmI.

Advantageously, the DNA used is single stranded DNA made partially double stranded by annealing one or more oligonucleotides.

In another attractive embodiment of the invention at least one probe comprises two separate probe parts being connected together in the step of connecting the essentially adjacent probes. “Probe parts” are herein defined as two nucleic acid sequence stretches that, once linked together, make up the probe. Said stretches may be of different length. Preferably, at least one of said probe parts comprises enzymatic template directed polymerised nucleic acid prior to said connecting. This embodiment can in one aspect be used to add a stuffer to the probes, resulting in a larger amplicon, whereas not all of said at least one probe needs to be generated through enzymatic template directed polymerisation prior to said connecting. This embodiment is elucidated in FIG. 12 of U.S. Pat. No. 6,955,901.

The relative or absolute abundance of the target nucleic acid can also be quantified, in parallel with the MS-MLPA technique. In order to do so, a sample is taken from the reaction mixture, wherein step 4), i.e. the digestion by the methylation sensitive restriction endonuclease, is omitted, therewith providing a straight-forward MLPA approach for the said quantification. Both a single or multiple single stranded target nucleic acids can be quantified this way, by using appropriate probe sets. Further, a control sample can be taken from the reaction mixture, wherein no methylation sensitive restriction enzyme is added, in order to check the maximum performance of the system, as in that case, all the target nucleic acid, both methylated and unmethylated, will be amplified. As another control, MS-MLPA reactions can be performed in parallel, wherein in a control unmethylated target nucleic acid is provided to check the proper activity of the restriction enzyme(s).

A further application of the current invention is the detection of pathogens in a sample. There are many different pathogens that can contaminate food samples or be present in clinical samples. Determination of even minor quantities of a pathogen can be accomplished using nucleic acid amplification methods such as PCR, RT-PCR and 3SR. However, for these purposes, considering the wide variety of potential pathogens, a large number of different primer sets need to be used and their performance optimised. Although possible, this is a lengthy process. In addition, very often not all primer sets can be added in one reaction mix thus necessitating different reactions for full coverage of the potential pathogens. With the present invention it is possible to scrutinise the presence or absence of a large number of different pathogens in a sample. This can be accomplished by analysing methylated DNA in a sample.

In another aspect, the invention further provides a nucleic acid probe for use in the claimed method, comprising a single stranded sequence, constituting one of the strands of the double stranded recognition site of the methylation sensitive restriction enzyme. Such a probe can be used according to the invention; by hybridizing to a complementary sequence in a target nucleic acid, a double-stranded recognition site for the methylation sensitive restriction enzyme is created, enabling the said restriction enzyme to cleave, if the target nucleic acid is not methylated.

In another embodiment, the nucleic acid probe comprises at least at one of the termini thereof, at least a part of a single stranded sequence, constituting one of the strands of a double stranded recognition site of the methylation sensitive restriction enzyme. Such a probe can still provide for one of the strands of a double-stranded recognition site for the methylation sensitive restriction enzyme, when the said terminus is connected, by the method according to the invention, with the terminus of another probe, providing the missing portion of the said recognition sequence. By connecting (ligation) of the said probes, the required recognition sequence is created, and the double stranded probe assembly, formed with the said probes in the claimed method, provides the double-stranded recognition site for the methylation sensitive restriction enzyme.

Preferably, the probe comprises enzymatic template directed polymerised nucleic acid.

In another aspect the invention provides a mixture of nucleic acids comprising two or more probes as described above. Again, at least one of these preferably comprises enzymatic template directed polymerised nucleic acid.

In such a probe mixture of at least a first nucleic acid probe and a second nucleic acid probe and optionally a third nucleic acid probe as defined above, one of the strands of a double stranded recognition site of the methylation sensitive restriction enzyme is formed when the 3′ end of the first probe is connected to the 5′ end of the second probe, or, if the third probe is present, the 3′ end of the first probe is connected to the 5′ end of the third probe or the 3′ end of the third probe is connected to the 5′ end of the second probe.

In yet another aspect the invention provides a kit for performing a method of the invention, comprising a nucleic acid probe, or a mixture of such probes as discussed above. Preferably, the kit also comprises liquid medium, preferably containing the at least one probe in a concentration of 20 nM or less. With such a kit, the probes are provided in the required low amount to perform reliable multiplex detection reactions according to the present invention.

In another embodiment, a kit for performing the method according to the invention is provided, the kit comprising a nucleic acid probe comprising enzymatic template directed polymerised nucleic acid, or a probe mixture comprising at least one of such probes.

In still another aspect, the invention provides a kit comprising a thermostable ligation enzyme of the invention, optionally further comprising a nucleic acid probe and or a mixture of probes according to the invention.

In still another aspect the current invention provides a series of related viral or plasmid cloning vectors that can be used to prepare probes for use in the current invention and having different stuffer sequences.

In the current invention not the target nucleic acids present in the sample are amplified, but (ligated) oligonucleotide probes provided to the sample. Target nucleic acid sequences originally found in the sample being analysed are not amplified because such target sequences do not contain amplification primer-specific tags.

In several of our examples we have obtained labelled amplification products by using a fluorescent primer and have separated the amplification products using an acrylamide based gel electrophoresis system with a one colour fluorescent detection system. Some automatic sequenators rely on the use of four differently fluorescently labelled primers each having a unique colour signature, enabling the analysis of more than one sample in a single lane and the use of internal size standards. It is however also possible to use PCR primers which are radioactively labelled, or that are labelled with other compounds that can be detected with the use of the appropriate calorimetric or chemiluminescent substrates. In a clinical setting and for general use in many clinical testing laboratories, it is preferable that methods not requiring the use of radiolabeled nucleotides be used.

In another preferred embodiment, mass spectrometry is used to detect and identify the amplification products.

In a third preferred embodiment, the melting temperature of the amplification products which can be influenced by the choice of the stuffer fragment is used to identify the amplification-products.

In a fourth preferred embodiment, the presence of a sequence tag on the amplification products is used to detect the amplification products and to analyse the results of the experiment. A sequence tag can easily be incorporated in the stuffer region of the probes and can be used to discriminate e.g. probes specific for wild-type sequences and probes specific for mutant sequences. Separation of the fragments by gel electrophoresis is not necessary as the use of fluorogenic probes and the use of the 5′ nuclease activity of some polymerases that can be used in the amplification reaction permits real time quantitative detection of the formation of at least two different sequence tags for instance one tag specific for a control wild-type specific probe and the other tag being specific for one or more different mutant sequences.

The necessary fluorogenic probes are described for instance by Lee et al (Nucleic Acid Research 21: 3761-3766 1993). Detection of fluorescence during the thermal cycling process can be performed for instance with the use of the ABI Prism 7700 sequence detection System of the PE Biosystems Corp. Other real time detection methods that do not rely on the destruction of sequence tag bound oligonucleotides by the 5′ nuclease activity of a polymerase but on the increased fluorescence of some fluorogenic probes (molecular beacons) upon binding to the sequence tag can also be used in the present invention as well as detection probes consisting of two entities, each being complementary to sequences present on one or more amplification-products and each containing a fluorescent moiety wherein fluorescent resonance energy transfer (FRET) occurs upon binding of both entities to the target amplification product.

Although the preferred embodiment of the invention uses the polymerase chain reaction for amplification of the probes used, other amplification methods for nucleic acids such as the 3SR and NASBA techniques are also compatible with the current invention.

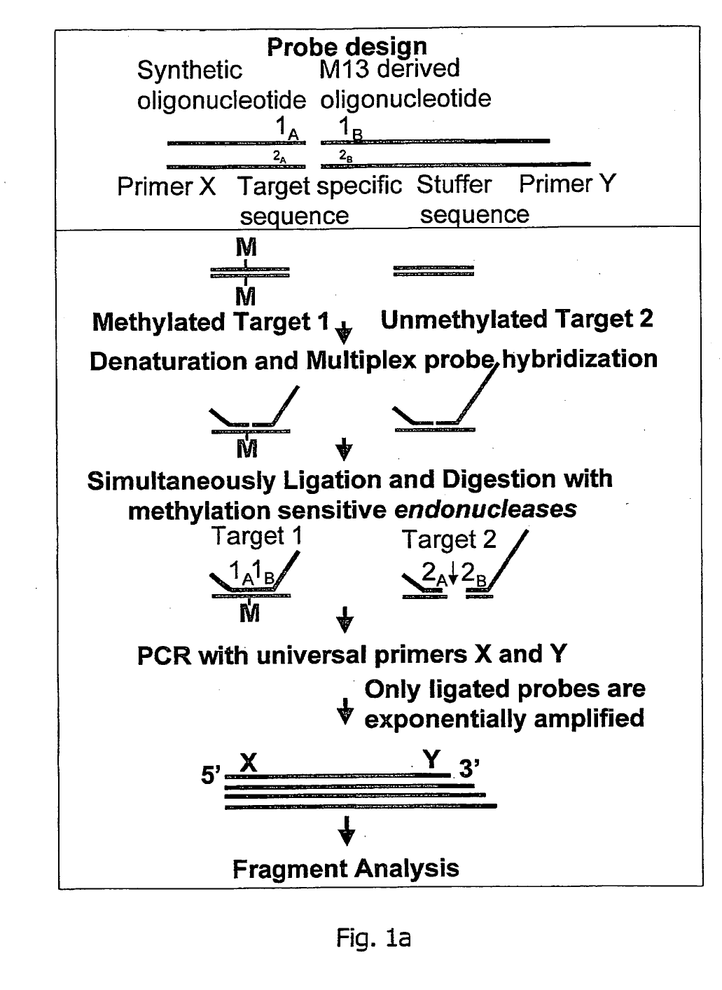

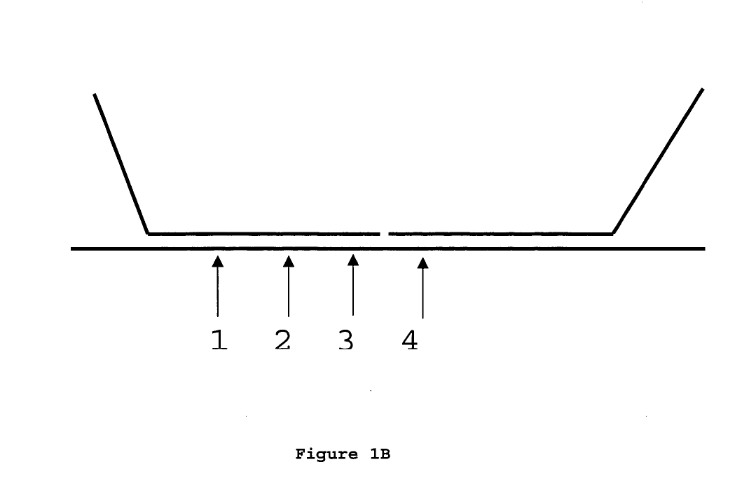

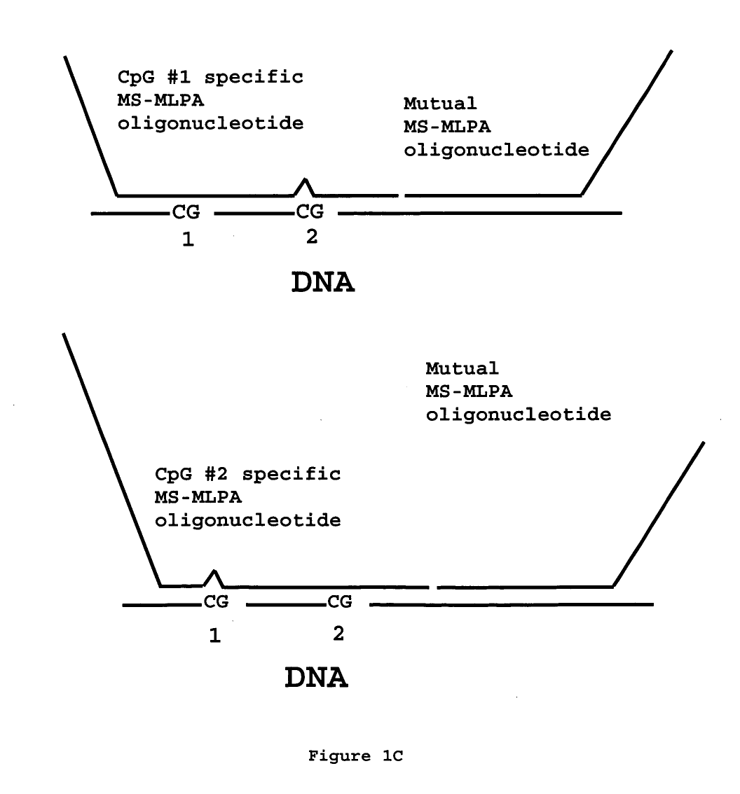

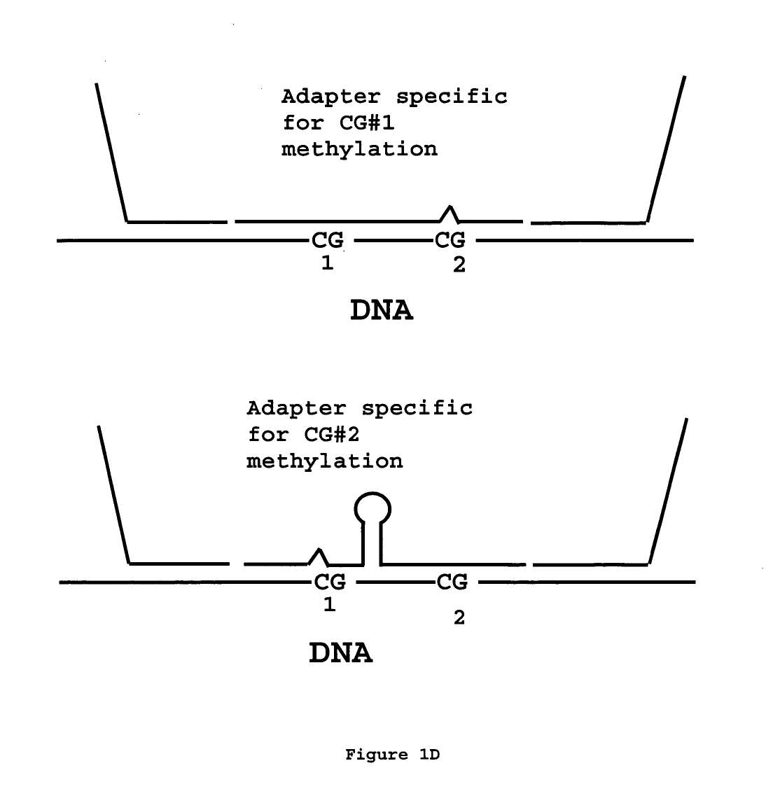

An outline of the method described in the current invention is shown in FIG. 1. It is to be noted that specific reference is made to U.S. Pat. No. 6,955,901, wherein the MLPA technique is described and explained by numerous examples and drawings, which are deemed to be incorporated by reference herein.

The method described herein is referred to as Methylation Specific Multiplex Ligatable Probe Amplification (MS-MLPA).

More in detail, and more exemplified by the examples below, FIG. 1A outlines the MS-MLPA procedure. An ordinary MLPA probe harbors two oligonucleotides, one short synthetic and one long M13-derived oligonucleotide and up to 50 probes can be added to each MLPA reaction. Both oligonucleotides contain universal primers sites. For each MLPA probe, the M13 oligonucleotide is cloned in a M13-vector that contains stuffer sequence that varies in length between the different probes. Subsequently, these long M13-oligonucleotides are obtained by restriction-digestion from the M13 clones. For MS-MLPA, the probe design is similar to an ordinary MLPA probe except that the sequence detected by the MS-MLPA probe contains a recognition sequence for a methylation sensitive endonuclease. Upon digestion of the DNA/MS-MLPA probe complex with a methylation sensitive enzymes, probes of which the recognition sequence is methylated will generate a signal. If the CpG_site is unmethylated the genomic DNA/MS-MLPA probe complex will be digested and prevent exponential amplification and no signal will be detected after fragment analysis.

In FIG. 1B, the arrows indicate the recognition sequences for different methylation sensitive restriction endonucleases. In a first reaction, aberrant methylation is detected using a combination of different methylation sensitive restriction endonucleases and MS-MLPA probes. In additional reactions 2-5, specific methylation is detected using different methylation sensitive restriction endonucleases in different reactions, that also serve as controls and verification of aberrant methylation for each other.

In FIG. 1C, for detection of specific methylation, a oligonucleotide containing mismatches for all the uninformative CpG sites is added to the reaction, this mismatched recognition sequence (ˆ) will not be digested. It is to be observed that if the cytosine (C) of the perfectly hybridized site is not methylated, the probe will be digested and no MS-MLPA specific exponential amplification will occur in the subsequent PCR reaction. A number of different synthetic oligonucleotides can thus be added to the reaction, each specific for different CpG sites and a common m13 derived oligonuclotide. As all the added oligonucleotides are of different length, respective methylation can be identified by fragment analysis

In FIG. 1D, the probe set comprises a third probe, wherein mismatched recognition (ˆ) and palindrome formation can take place. It is again to be observed that if the cytosine (C) is not methylated and the probe is perfectly hybridized., the probe will be digested and no MS-MLPA specific exonential amplification will occur in the subsequent PCR reaction. Mismatched recognition sequence (ˆ) will not be digested. A number of these synthetic oligonucleotides can thus be added to the reaction, each specific for different CpG sites and a common m13 derived oligonuclotide. As all the added oligonucleotides are of different length, respective methylation can be identified by fragment analysis

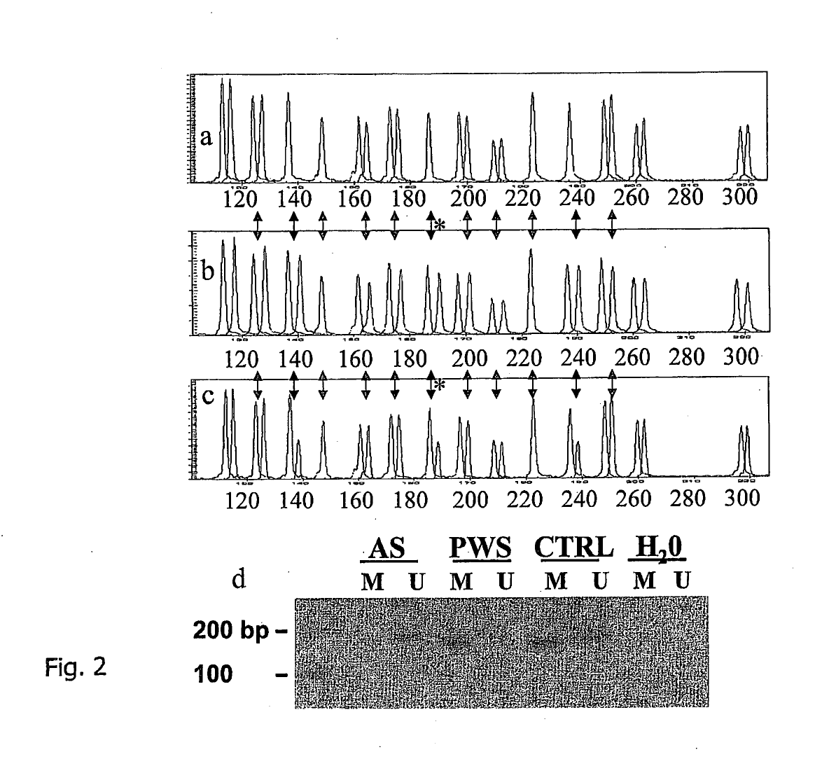

FIG. 2 shows the detection of the methylation status of the imprinting center in chromosome 15 by MS-MLPA. Approximately 100 ng of DNA from patients diagnosed with either PWS or AS and control DNA from healthy persons was subjected to MS-MLPA using the ME028 PWS/AS probe mix. Only a part of the capillary electrophoresis (CE) pattern is shown. Blue signals correspond with undigested DNA/probe complex. Red signals correspond with the same samples but after digestion of the DNA/probe complex with HhaI. Black arrows indicate peaks generated from three different MS-MLPA probes in the promoter region of the SNRPN gene. The star indicates the probe for which MSP was performed. Red arrows indicate places for two MS-MLPA probes; one within the chromosome 15q11 imprinted center and one outside that are not methylated and serve as controls for proper digestion. Blue arrows correspond with other probes located in the chromosome 15q11 region without a HhaI site that serves as a control for the copy number quantification. a, CE pattern from an AS patient which has both alleles unmethylated therefore after MS-MLPA no signal is generated from the MS-MLPA probes. b, CE pattern generated from a PWS patient. Patients diagnosed with PWS due to uniparental disomy inherit only the maternally methylated allele in the promoter of the SNRPN gene, thus both alleles are methylated and therefore will generate a normal signal. c, CE pattern from control DNA. Normal individuals have one methylated and one unmethylated allele, thus a 50% reduction of the signal is seen. d, MSP results on the three samples from above confirming the MS-MLPA results for the region recognized by the 166 bp SNRPN promoter specific MS-MLPA probe.

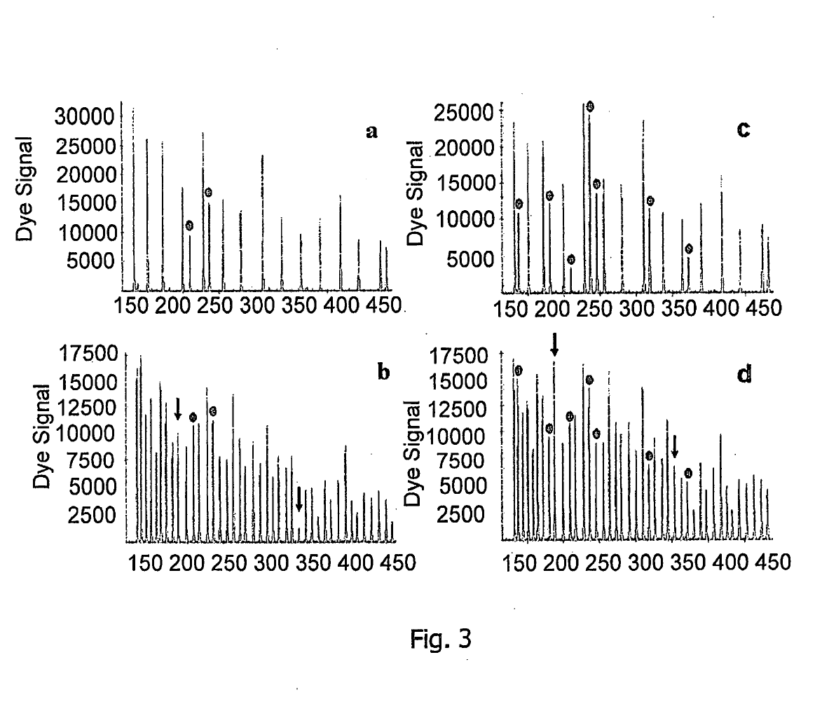

FIG. 3 shows the detection of aberrant methylation patterns in AML cell lines by MLPA using the P041A probe mix. a, CE-pattern from an AML cell line showing 50% methylation of p15 (211 bp) and p73 (238 bp) genes (red dots). Total absence of all other MS-MLPA probes indicates 100% efficiency in the digestion reaction. b, CE-pattern from the same cell line (as depicted in figure a) but without HhaI digetion of the DNA/probe complex, showing the undigested peak heights that were used for quantification of the methylation levels. When compared to control DNA samples, a reduced probe signal specific for the MEN1 and HIC1 promoters is seen as depicted by the black arrows, indicating a decrease in copy number of these genes (FIG. 3b, 193 and 355 bp fragments). The expected normal probe signals specific for the MEN1 and HIC1 promoters is depicted by black arrows in FIG. 3d. c, CE-pattern from an AML cell line showing methylation of several genes including TIMP-3 (142 bp), KLK10 (184 bp), p15 (211 bp), p73 (238 bp), CDH13 (247 bp), IGSF4 (319 bp) and ESR1 (373) (red dots). d, CE-pattern from the same cell line (as depicted in figure c) but without HhaI treatment.

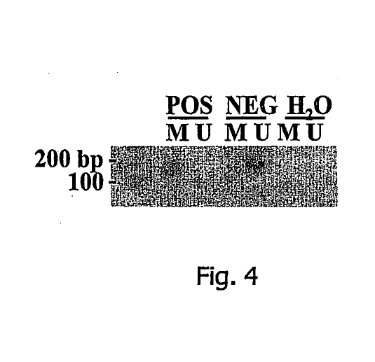

FIG. 4 shows methylation specific PCR (MSP). MSP using specific primers to amplify the p15 promoter region of two AML samples; one AML sample that showed positive methylation of the p15 gene with MS-MLPA (POS) and one AML sample that was negative with MS-MLPA (NEG). Also a MSP is included with only H₂O as a control. As expected a PCR product of 160 bp was detected with primers designed to amplify methylated sequences (M) in the AML cell line positive for p15 methylation. In the AML cell line that was negative for p15 methylation with MS-MLPA only a 169 bp PCR product was detected indicating that this cell line was indeed not methylated for the p15 gene.

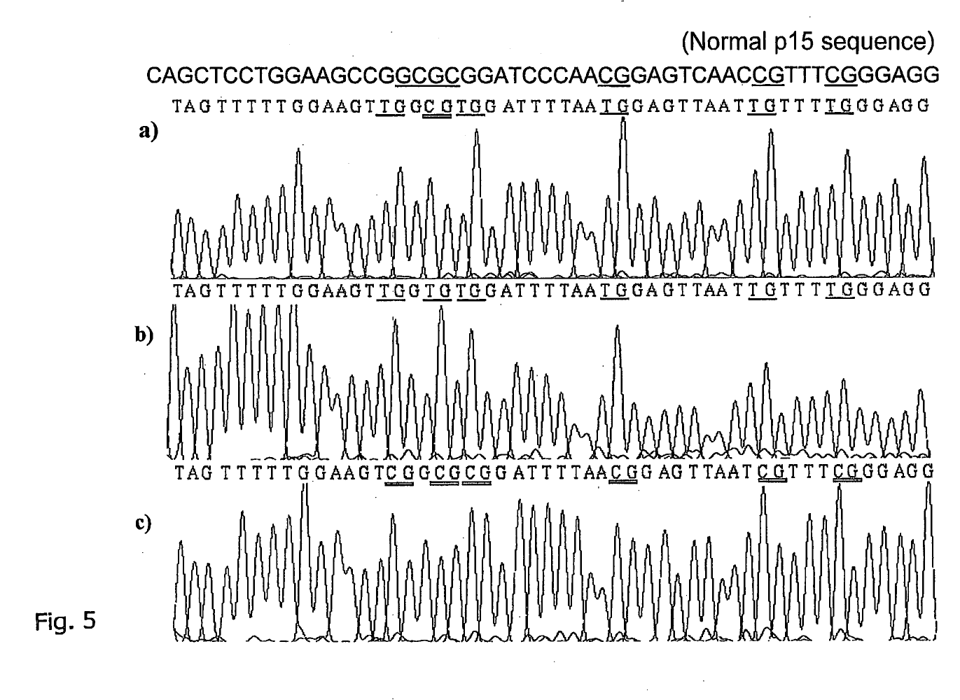

FIG. 5 shows the methylation status of p15 promoter region recognized by MS-MLPA being analyzed by bisulphite DNA sequencing. Three samples were sequenced a, One control DNA sample treated with HhaI methylase, b, One DNA sample of an AML cell line that was negative for p15 methylation after MS-MLPA and c, One AML cell line that was positive for p15 methylation after MS-MLPA. The HhaI site recognized by the MS-MLPA probe is double underlined. All the CpG sites in this region are indicated (underlined). On top, part of the DNA sequence of the normal p15 sequence (without bisulphite treatment) is depicted.

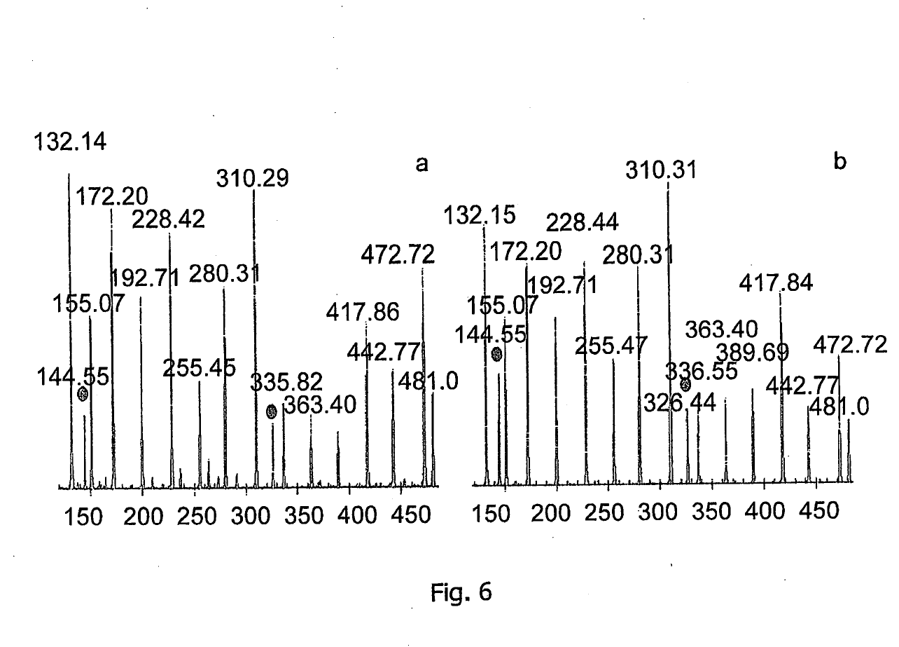

FIG. 6 shows a comparison of MS-MLPA reactions performed on DNA obtained from the same breast tumors that were either paraffin embedded or fresh-frozen. Samples were analyzed using the P041A probe mix. Indistinguishable MS-MLPA results were obtained with DNA from paraffin embedded or fresh frozen tumor tissues. a, CE-pattern from a MS-MLPA performed on DNA extracted from paraffin-embedded tissue. Red dots indicate methylation of one of the alleles of the APC promoter (148 bp) and the ESR1 promoter (373 bp). b, CE-pattern from a MS-MLPA performed on DNA from the same sample but derived from fresh-frozen material, showing the same methylation pattern.

Having now generally described the invention, the same will be more readily understood through reference to the following example which is provided by way of illustration and is not intended to be limiting of the present invention.

### EXAMPLE

DNA Samples

DNA samples of 16 anonimized patients diagnosed with PWS or AS were kindly provided by Ans van den Ouweland, Erasmus MC, Rotterdam, The Netherlands.

Genomic DNA was isolated from 21 AML cell lines of patients that had high blast counts. Tumor DNA samples, either paraffin-embedded or fresh-frozen, were kindly provided by Petra Nederlof, Netherlands Cancer Institute, NKI-AvL, Amsterdam, The Netherlands.

Methylated DNA was obtained by treating human genomic DNA (Promega) with HhaI methylase (New England Biolabs) in the presence of S-adenosylmethionine according to the manufacturer's instructions.

Paraffin-Embedded DNA Extraction

Slides with a slice of paraffin-embedded tissue (5 mm×5 mm, lolm of thickness) were heated for 15 min at 75° C. to melt the paraffin. The hot slides were placed in Xylol for 5 min. This was repeated until the paraffin oil was completely dissolved. The slides were then incubated for 30 seconds periods in 99%, 96%, 75% ethanol, tap water and finally placed in 1M NaSCN at 37° C. overnight. The next day the slides were washed with TE-Buffer (10 mM Tris-HCl pH 8.5, 1 mM EDTA) and air dried. A few drops (20-40 μl) Proteinase K solution (2 mg/ml recombinant Proteinase K (Roche) in 25 mM Tris-HCl pH 8.2) were applied onto the tissue. The tissue was transferred to a 1.5 ml tube containing 100 μl Proteinase K solution and incubated overnight at 37° C. After 20 min incubation at 80° C. to inactivate the Proteinase K, the tubes were centrifuged for 10 min at 13,000 rpm on a table centrifuge. Finally, 2 μl of the supernatant was used for each MS-MLPA reaction.

MLPA Probe Design

The design of the MS-MLPA probes was performed as described by Schouten et al., 2002. However, each probe used in this study for methylation quantification analysis contained one HhaI restriction site in the target recognition sequence. In this study three probe mixes were developed, the P028 PWS/AS, the P041A, and the P041B mix. The P028 PWS/AS probe mix contains 25 probes specific for most of the genes in the PWS/AS critical region of chromosome 15q11-q13 and two probes for genes that are located outside this region. Among the probes in the critical region 10 probes contain a HhaI recognition site. Furthermore, 14 control MLPA and MS-MLPA probes are included that are not specific to genes on chromosome 15 (for details see Table 1).

The methylation mix, P041A, contains a panel of 41 probes specific to 22 tumor suppressor genes (for further details see Table 2). The mix contains for 19 genes a single probe that detects a HhaI sequence within the promoter region of these genes. For VHL and CDKN2A, two probes are included. For the promoter region of MLH1, three probes are included. The remaining 15 probes in this mix lack a HhaI sites in their recognition sequence and serve as control probes. These probes are used for quantification of the methylation levels. The P041B mix contains MS-MLPA probes for the same genes detected by the P041A mix, except that these probes recognize a different CpG site in the corresponding promoter regions. Details on probe sequences, gene loci, and chromosome locations can be found at www.mlpa.com.

TABLE 1
The P028 PWS/AS probe mix

| 1Size | | Chr. | |
|---|---|---|---|
| (bp) | Gene | Pos. | Recognition sequence |
| 130 | IL4 | 05q31.1 | CTACATTGTCACTGCAAATCGACACCTAT-TAATGGGTCTCACCTCCCAACTGCTTCCCCCT |
| 136 | CYFIP1 | 15q11 | GAGCTGGATGGCCTGTTGGAAATC-AACCGCATGACCCACAAGCTGCTGAGCCGGTACCTGACGCTG |
| 142 | SNRPN | 15q12 | 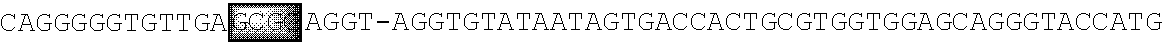 |
| 148 | BRCA2 | 13q12.3 | 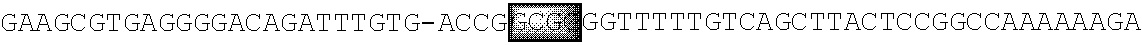 |
| 154 | KIAA1899 | 15q11.1 | GGACCAACACTTGTACAGCAGTGATCCATT-GTATGTTCCAGATGACAGGGTTTTGGTTACTGAGACTCAGGTT |
| 160 | UBE3A | 15q12 | GAAGAAGACTCAGAAGCATCTTCCTCAAGG-ATAGGTGATAGCTCACAGGGAGACAACAATTTGCAAAAATTAG |
| 166 | SNRPN | 15q12 | 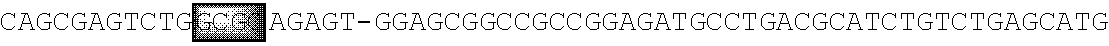 |
| 172 | MKRN3 | 15q11.2 | GCGACATGTGTGGGCTGCAGACCTT-GCACCCCATGGATGCTGCCCAGAGGGAAGAACATATCATG |
| 178 | MAGEL2 | 15q11.2 | 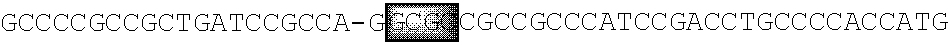 |
| 184 | UBE3A | 15q12 | 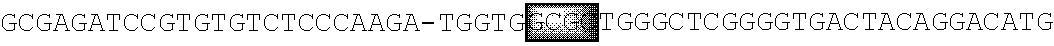 |
| 190 | SNRPN | 15q12 | 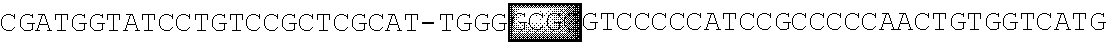 |
| 196 | MAGEL2 | 15q11.2 | CTCTGCTGCCTCAGAGACCCCAAA-GTCACTGCCATATGCTCTGCAGGATCCCTTTGCCTGTGTA |
| 202 | APBA2 | 15q12 | CAAAGGGTGTGCCCTCACCACCCACTT-GATTTTTTTCATTTTGCCAAAAAGGGGTATGTCTTTATCAAAG |
| 220 | GABRB3 | 15q12 | CTCAGGCGGCATTGGCGATACCAGGAA-TTCAGCAATATCCTTTGACAACTCAGGAATCCAGTACAGGAAA |
| 229 | SNRPN | 15q12 | 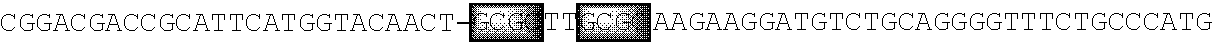 |
| 238 | PARK2 | 06q26 | GCGTGCACAGACGTCAGGTAAGGATCTAAA-AATAGTGTCACTTCCCTCCACGGACGTGAGGTAAGGATCTCATG |
| 247 | SNRPN | 15q12 | 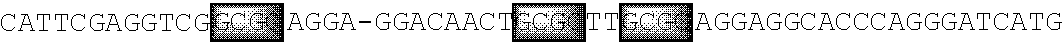 |
| 256 | SNRPN | 15q12 | GTTTGGTTTGCGGCAGAAGAATCTGCA-TTTCGAACAAGTGCCAGGACTGGTCTGAGGAACACACGTT |
| 265 | GATA3 | 10p15.1 | CGGCAGGACGAGAAAGAGTGCCTCAAG-TACCAGGTGCCCCTGCCCGACAGCATGAAGCTGGAGT |
| 274 | ATP7B | 13q14.2 | GACGCTGTCAAGCAGGAGGCT-GCCCTGGCTGTGCACACGCTGCAGAGCATGGCATG |
| 283 | FANCD2 | 03p25.3 | GGTGTGGCCAAGTGGGGATAAAGA-GAAGAGCAACATCTCTAATGACCAGCTCCATGCTCTGCTCCATG |
| 292 | SNRPN | 15q12 | GGTTTTTGCTTGGAATCAGATTCCTCGCTA-CTCCAATATGGCTTTAACCACCTCTTGGTGTCTCAGCTAAGAA |
| 310 | NF2 | 22q12 | GGCAGATCAGCTGAAGCAGGA-CCTGCAGGAAGCACGCGAGGCGGAGCGAAGAGCCAAGCAG |
| 319 | NDN | 15q11.2 | GCTAGTCCTCAGAGACACTGCTGCGA-GGGTAGTGGGCAGTGGGATTAGCCTCCCGCAGAGCCATG |
| 328 | BRCA1 | 17q21 | GATGCACAGTTGCTCTGGGAGTCT-TCAGAATAGAAACTACCCATCTCAAGAGGAGCTCATTAAG |
| 337 | DLEU1 | 13q14.3 | CAGGAGGTTGTTTGCTGTACTCTCCCTTGT-ACAGTTAGCTGTCTCTAGTGCCTGAATGCACTAATTGTCCTTT |
| 346 | BLM | 15q26.1 | 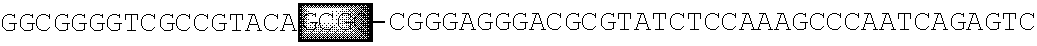 |
| 355 | UBE3A | 15q12 | GGAGTTCTGGGAAATCGTTCATTCATTTAC-AGATGAACAGAAAAGACTCTTCTTGCAGTTTACAACGGGCACA |
| 364 | ATP10A | 15q12 | 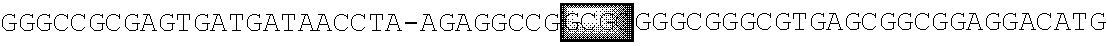 |
| 373 | SNAP29 | 22q11.21 | TGCAGACAGAAATTGAGGAGCAAGATG-ACATTCTTGACCGGCTGACAACCAAAGTGGACAAGTTAGATGT |
| 382 | ATP10A | 15q12 | GCGTCTTCGCTGCAACGAAATCTT-CCCTGCGGACATTCTGCTGCTCTCCTCCAGTGACCCCCATG |
| 391 | NFKBIA | 14q13 | TCAGGAGCCCTGTAATGGCCG-GACTGCCCTTCACCTCGCAGTGGACCTGCAA |
| 400 | UBE3A | 15qq2 | 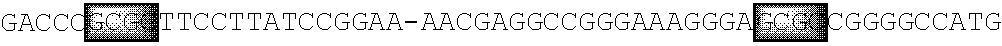 |
| 409 | GABRB3 | 15q12 | 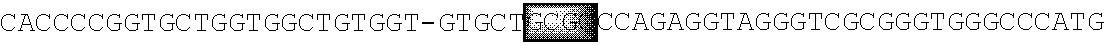 |
| 418 | NDN | 15q11.2 | 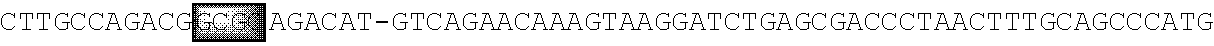 |
| 427 | MGC3329 | 17p13.3 | GGAGCTTCGCTATGCGGCTGCTTTAA-GATTCTAGGGTTGTACAGGCCCACGCCAGACACGACGTCTGCATG |
| 436 | OCA2 | 15q12 | CCCGCACCGCCGCTCATGTAT-GCCCTGGCCTTCGGTGCTTGCCTGGGAGGTAAGGCATG |
| 445 | OCA2 | 15q12 | 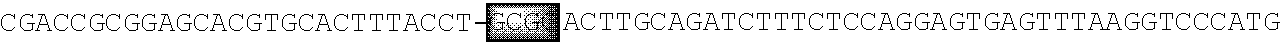 |
| 454 | IGF1R | 15q26 | CCCAGTCTTCGACCTGCTGAT-CCTTGGATCCTGAATCTGTGCAAACAGTAACGTG |
| 463 | MLH1 | 03p22.1 | 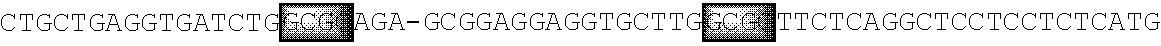 |
| 472 | NRXN1 | 02p16.3 | GAGTCGAAACTACATCAGTAACTCAGCACA-GTCCAATGGGGCTGTTGTAAAGGAGAAACAACCCAGCAGTCATG |

The size of the expected PCR products, the corresponding genes, probe names, chromosomal location and the recognition sequences are depicted. Within the recognition sequence the Hhal site (GcGc) as well as the ligation site are depicted (-) . The P028 PWS/AS probe mix contains 16 MS-MLPA probes specific for most of the genes in the PWS/AS critical region of chromosome 15. In addition, 18 control probes that are located outside the critical region and on other chromosomes are included.

TABLE 2
P041A probe mix

| Size | | Chr. | |
|---|---|---|---|
| (bp) | Gene | Pos. | Recognition sequence |
| 136 | CREM | 10p12.1 | GCTCCTCCACCAGGTGCTACAAT-TGTACAGTACGCAGCACAATCAGCTGATGGCACACAGCAGT |
| 142 | TIMP3 | 22q12.3 | 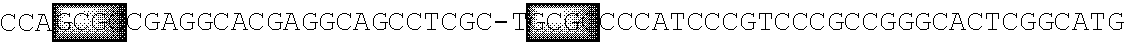 |
| 148 | APC | 05q22 | 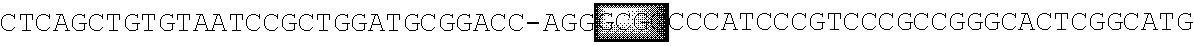 |
| 154 | IL4 | 05q31.1 | CATTGTCACTGCAAATCGACACCTAT-TAATGGGTCTCACCTCCCAACTGCTTCCCCCT |
| 160 | CDKN2A | 09p21 | 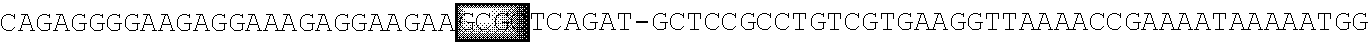 |
| 166 | MLH1 | 03p22.1 | 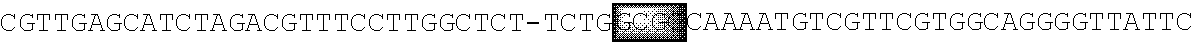 |
| 175 | TNFRSF1A | 12p13 | GCCACACTGCCCTCAGCCCAA-ATGGGGGAGTGAGAGGCCATAGCTGTCTGGC |
| 184 | KLK10 | 19q13.3 | 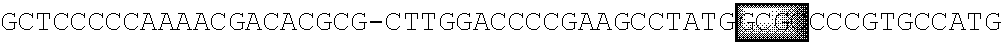 |
| 193 | MEN1 | 11q13 | 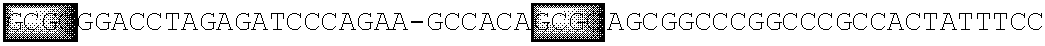 |
| 202 | MLH3 | 14q24.3 | GCGACCTTGTTCTTCCTTTCCTTCCGA-GAGCTCGAGCAGAGAGGACTGTGATGAGACAGGATAACAG |
| 211 | CDKN2B | 09p21 | 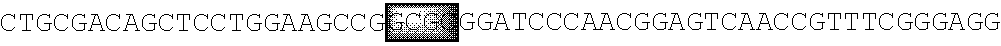 |
| 220 | VHL | 03p25.3 | 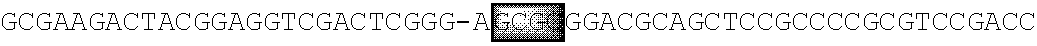 |
| 229 | NF2 | 22q12 | GGGATGAAGCTGAAATGGAATATCTGAAG-ATAGCTCAGGACCTGGAGATGTACGGTGTGAACTACTTTGCAATCCG |
| 238 | TP73 | 01p36 | 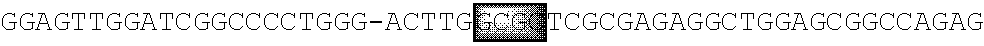 |
| 247 | CDH13 | 16q24.2 | 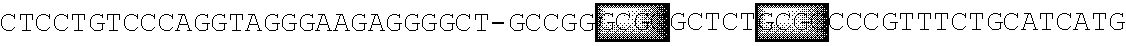 |
| 256 | BCL2 | 18q21.3 | CTTCTCCTGGCTGTCTCTGAAGACTC-TGCTCAGTTTGGCCCTGGTGGGAGCTTGCATC |
| 265 | FANCD2 | 03p25.3 | 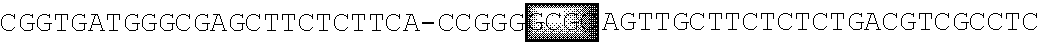 |
| 274 | VHL | 03p25.3 | 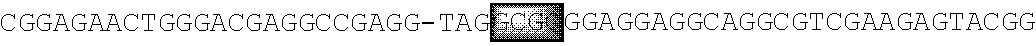 |
| 283 | TSC2 | 16p13.3 | GAGCCAGAGAGAGGCTCTGAGAAGAAG-ACCAGCGGCCCCCTTTCTCCTCCCACAGGGCCTCCTGCATG |
| 292 | MLH1 | 03p22.1 | 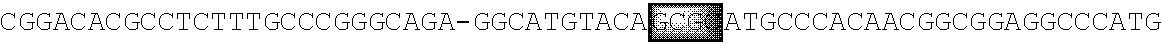 |
| 301 | BRCA2 | 13q12.3 | 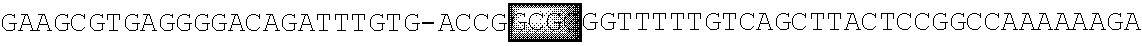 |
| 310 | RB1 | 13q14.2 | CGTGAGTTTTAGACAAGCTAGCTTTTGTGTTG-TCTTGGCGGCCATATTTGTAAGAAGGGTGAGAAGTATG |
| 319 | IGSF4 | 11q23 | 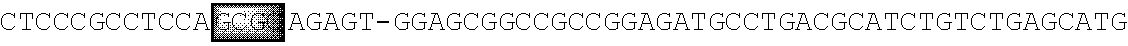 |
| 328 | RASSF1 | 03p21.3 | 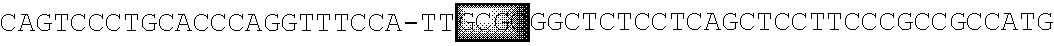 |
| 337 | BRCA1 | 17q21 | CCCTTACCTGGAATCTGGAATCAG-CCTCTTCTCTGATGACCCTGAATCTGATCCTTCT |
| 346 | DAPK1 | 09q22 | 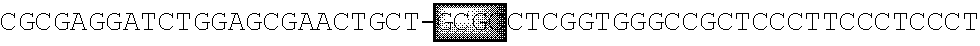 |
| 355 | HIC1 | 17p13.3 | 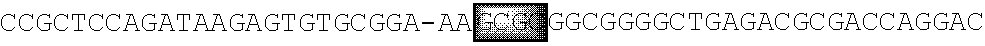 |
| 364 | MSH6 | 02p16 | CGCCTGAACAGCCCTGTCAAAGTT-GCTCGAAAGCGGAAGAGAATGGTGACTGGAAATGGCTCTC |
| 373 | ESR1 | 06q25.1 | 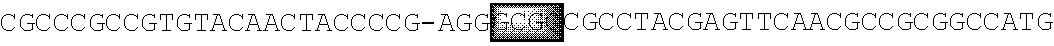 |
| 382 | CDKN1B | 12p13.2 | 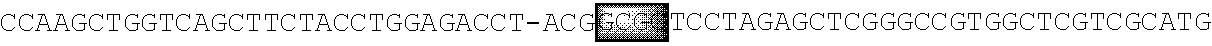 |
| 391 | KLK3 | 19q13 | TGTGTCACCATGTGGGTCCCG-GTTGTCTTCCTCACCCTGTCCGTGACGTGGA |
| 400 | ASC | 16p12 | 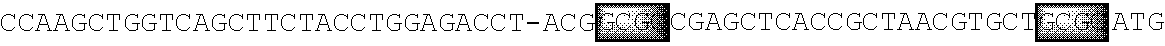 |
| 409 | FHIT | 03p14.2 | CGCGGGTCTGGGTTTCCACGC-GCGTCAGGTCATCACCCCGGAGCCCAGTGGGCATG |
| 418 | BRCA2 | 13q12.3 | GGCCATGGAATCTGCTGAACAAAA-GGAACAAGGTTTATCAAGGGATGTCACAACCGTGTGGAAGTTGCGT |
| 427 | CDKN2A | 09p21 | 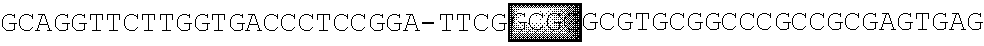 |
| 436 | BRCA1 | 17q21 | 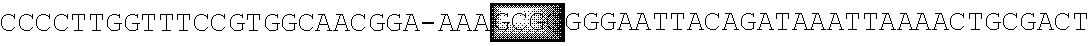 |
| 445 | TNFRSF7 | 12p13 | GAAAGTCCTGTGGAGCCTGCA-GAGCCTTGTCGTTACAGCTGCCCCAGGGAGG |
| 454 | GSTP1 | 11q13 | 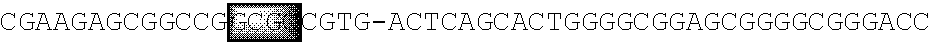 |
| 463 | MLH1 | 03p22.1 | 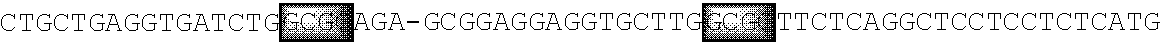 |
| 472 | NRAS | 01p13.2 | TCTCAACAGCAGTGATGATGGGACTCA-GGGTTGTATGGGATTGCCATGTGTGGTGATGTAACAAGGTGAG |
| 481 | MFHAS1 | 08p23.1 | CACTTACGACGCCTTCGGGACAAGTT-GCTGTCAGTTGCTGAGCACCGAGAGATCTTCCCCAACTTAC |
| The size of the expected PCR products, the corresponding genes, probe names, chromosomal location and the recognition sequences are depicted. Within the recognition sequence the Hha1 site (GCGC) as well as the ligation site are depicted (-). The P041A probe mix contains one probe specific for each promoter region of the 19 genes. For 2 genes (VHL and CDKN2A) two probes are included and for the promoter region of MLH1, three probes are included. The | | | |
| # remaining 15 probes in this mix lack a HhaI sites in their recognition sequence and serve as control probes. | | | |

MS-MLPA Assay

MLPA reagents were obtained from MRC-Holland, Amsterdam, The Netherlands (EK1 kit; www.mlpa.com). Approximately 25 ng of genomic DNA in 5 μl of TE-Buffer (10 mM Tris-HCl (pH 8.5), 1 mM EDTA) was denatured for 10 min at 98° C. SALSA MLPA buffer (1.5 μl) and MS-MLPA probes (1 fmol each, volume 1.5 μl) were then added and after incubation for 1 min at 95° C., allowed to hybridize to their respective targets for approximately 16 hours at 60° C. After hybridization, the mix was diluted at room temperature with H₂O and 3 μl Ligase buffer A to a final volume of 20 μl and then equally divided in two tubes. While at 49° C. a mix of 0.25 μl Ligase −65 (MRC-Holland), 5 U HhaI (Promega) and 1.5 μl Ligase buffer B in a total volume of 10 μl was added to one tube. The second tube was treated identical except that the HhaI enzyme was replaced with H₂O. Simultaneous ligation and digestion was then performed by incubation for 30 min at 49° C., followed by 5 min heat inactivation of the enzymes at 98° C. The ligation products were PCR amplified by addition of 5 μl of this ligation-mix to 20 μl PCR reaction-mix containing PCR buffer, dNTPs, SALSA polymerase and PCR primers (one unlabeled and one D4-labeled) at 60° C. as described by Schouten et. al. (2002).

Fragment and Data Analysis

Automated fragment and data analysis was performed by exporting the peak areas to an excel based analysis program. In brief, for copy number quantification, every sample peak area was divided by the nearest control peak areas. Relative copy number was obtained by comparing this ratio with the same ratio obtained from a control sample. Quantification of the methylation status of a particular CpG site was done by dividing the peak area with the combined areas of the control probes lacking a HhaI site. Finally, the relative peak area of each target probe from the digested sample was compared with those obtained from the undigested sample. Aberrant methylation was scored when the calculated methylation percentage was higher than 10%. Any methylation percentages below this level were regarded as background. All MS-MLPA reactions were performed at least three times.

Methylation Specific PCR (MSP) and Bisulphite Sequencing

The DNA samples were sodium bisulphite converted using the EZ DNA Methylation Kit (Baseclear, Netherlands) following the manufacturer's instructions. The modified DNA was amplified using methylated and unmethylated specific primers to amplify the same fragment within the promoters of SNRPN and p15 as the respective MS-MLPA probes. The PCR conditions were for all reactions a denaturation step at 95° C. for 5 minutes; 32 cycles at 95° C. for 40 s, 65° C. for 30 s, 72° C. for 60 s. Finally the PCR products were visualized on a 2.5% agarose gel.

For SNRPN the following methylated primers were used 5′-CGCGGTCGTAGAGGTAGGTTGGCGC and 5′GACACAACTAACCTTACCCGCTCCATCGCG resulting in a 167 bp product.

For the unmethylated reaction the following primers 5′-GTATGTTTGTGTGGTTGTAGAGGTAGGTTGGTGT and 5′-CACCAACACAACTAACCTTACCCACTCCATCACA resulting in a 180 bp product.

For MSP of p15 the following methylated primers were used 5′-GAAGGTGCGATAGTTTTTGGAAGTCGGCGC and GACGATCTAAATTCCAACCCCGATCCGCCG resulting in a 160 bp product. For the unmethylated reaction the following primers 5′-GTGGAGAAGGTGTGATAGTTTTTGGAAGTTGGTGT and 5′-CATCAACAATCTAAATTCCAACCCCAATCCACCA resulting in a 169 bp product.

For bisulphite sequencing of the p15 gene the following primers were used to amplify a 291 bp of the promoter region including the target sequences recognized by the p15 MS-MLPA probe : p15-forward 5′-TAGGTTTTTTAGGAAGGAGAGAGTG-′3 and p15-Reverse 5′-CCTAAAACCCCAACTACCTAAATC. Subsequently the nested forward primer: 5′-AGGAGAATAAGGGTATGTTTAGTGG-3′ was used for sequencing.

Results

MS-MLPA with Prader-Willy and Angelman Samples

To validate MS-MLPA, we used the P028 PWS/AS probe mix to analyze DNA samples of patients with PWS and AS. These syndromes are distinct neurogenetic disorders, which are characterized by deletions or uniparental disomy resulting in aberrant expression of genes located in the imprinted region on chromosome 15q11-q13. Absence of a paternal contribution of chromosome 15q11-q13, due to hemizygous deletion or uniparental disomy, results in PWS. The absence of the corresponding maternal copy of the same region causes Angelman syndrome (Lalande, M. (1996) Parental imprinting and human disease. Annu.Rev.Genet., 30:173-95. , 173-195). Among the probes in the P028 PWS/AS mix seven probes are specific to the SNRPN gene, which is located in the imprinting center. Five are MS-MLPA probes containing a HhaI restriction site. If the site is not methylated, HhaI digestion will prevent exponential amplification of the MS-MLPA probe (see FIG. 1A). Patient with AS due to uniparental disomy harbor two unmethylated alleles and accordingly no MS-MLPA signal is observed (FIG. 2a). DNA from a patient with the PWS syndrome due to uniparental disomy shows no differences in peak areas of the SNRPN specific MS-MLPA probes between the digested and undigested sample DNA (FIG. 2b), indicating that all CpG sites are methylated. DNA of control individuals shows a 50% reduction of the MS-MLPA signal, corresponding to the presence of one methylated allele (FIG. 2c).

Aberrant Methylation in AML Cell Lines

Acute myeloid leukemia is a heterogeneous disorder with regard to morphology and chromosome aberrations detected in the leukemic cells. Various genes known to be silenced by promoter methylation have been analyzed in AML. Frequent aberrant promoter methylation of the tumor suppressor genes p15^{INK4b}, p16^{INK4a}, and p73, has been described by different groups (Esteller, M. (2003) Profiling aberrant DNA methylation in hematologic neoplasms: a view from the tip of the iceberg. Clin.Immunol., 109, 80-88; Herman, J. G., Jen, J., Merlo, A. and Baylin, S. B. (1996) Hypermethylation-associated inactivation indicates a tumor suppressor role for p15INK4B. Cancer Res., 56, 722-727; Voso, M. T., Scardocci, A., Guidi, F., Zini, G., Di Mario, A., Pagano, L., Hohaus, S. and Leone, G. (2004) Aberrant methylation of DAP-kinase in therapy-related acute myeloid leukemia and myelodysplastic syndromes. Blood, 103, 698-700). To evaluate MS-MLPA, we analyzed DNA samples derived from 21 AML cell lines for promoter methylation using probe mix P041A that contains MS-MLPA probes for 22 different genes (see Table 2). Of the 21 AML samples, frequent aberrant methylation of the genes p15^{INK4b} and p73 occurred in nine (42.9%) and in ten (47.6%) samples, respectively. Aberrant methylation was also found in the following genes: IGSF4 (28.6%), TIMP-3 (23.8%), ESR1 (19.1%), FHIT (9.5%) and CDH13 (9.5%). Two examples of a MS-MLPA profile are shown in FIG. 3. In one sample of an AML cell line aberrant methylation of the p15 (211 bp amplification product) and the p73 (238 bp) gene was detected (FIG. 3a). In addition, a decrease in copy number of the MEN1 and HIC1 promoter is seen as depicted by the black arrows in this sample (FIG. 3b, 193 and 355 bp fragments). The other AML sample shows aberrant methylation of several genes including TIMP-3 (142 bp), KLK10 (184 bp), p15 (211 bp), p73 (238 bp), CDH13 (247 bp), IGSF4 (319 bp) and ESR1 (373 bp) (FIG. 3c). Also shown are the undigested MS-MLPA profiles that were used for quantification of the methylation levels (FIG. 3b, d). A summary of the MS-MLPA test results including standard deviation is shown in Table 3. MS-MLPA experiments have been performed at least three times.

TABLE 3
Determination of the MS-MLPA assay variation. MS-MLPA results performed on DNA of 6
AML samples are shown. Only the genes are depicted that show aberrant methylation in these
samples. Experiments have been repeated at least three times.

| | Sample 1 | | Sample 2 | | Sample 3 | | Sample 4 | | Sample 5 | | Sample 6 | |
|---|---|---|---|---|---|---|---|---|---|---|---|---|
| | Average | | Average | | Average | | Average | | Average | | Average | |
| | ratio | SD | ratio | SD | ratio | SD | ratio | SD | ratio | SD | ratio | SD |
| MS-MLPA | | | | | | | | | | | | |
| probes | | | | | | | | | | | | |
| CDKN2B | 0.48 | 0.04 | 0.22 | 0.02 | 0.22 | 0.03 | 0.68 | 0.01 | 0.46 | 0.05 | 0.63 | 0.04 |
| TP73 | — | — | 0.86 | 0.1 | 0.21 | 0.02 | 0.50 | 0.04 | — | — | 0.45 | 0.03 |
| CDH13 | — | — | 0.77 | 0.09 | — | — | — | — | — | — | — | — |
| IGSF4 | — | — | 0.55 | 0.08 | 0.38 | 0.03 | — | — | — | — | 0.66 | 0.18 |
| ESR1 | 0.36 | 0.08 | 0.42 | 0.01 | 0.66 | 0.03 | 0.63 | 0.13 | — | — | — | — |
| FHIT | — | — | — | — | — | — | 0.51 | 0.11 | 0.26 | 0.08 | — | — |
| Control | | | | | | | | | | | | |
| Probes | | | | | | | | | | | | |
| CREM | 0.9 | 0.07 | 0.89 | 0.14 | 0.93 | 0.11 | 0.93 | 0.11 | 0.96 | 0.06 | 0.99 | 0.05 |
| PARK2 | 1.03 | 0.01 | 1.02 | 0.11 | 1.13 | 0.17 | 1.13 | 0.11 | 1.15 | 0.02 | 1.12 | 0.14 |
| TNFRSF1A | 0.9 | 0.1 | 0.9 | 0.09 | 0.97 | 0.04 | 0.97 | 0.01 | 0.98 | 0.05 | 0.95 | 0.05 |
| MLH3 | 1.08 | 0.02 | 1.08 | 0.07 | 1.23 | 0.06 | 1.11 | 0.10 | 1.07 | 0.04 | 1.2 | 0.01 |
| BCL2 | 1.04 | 0.07 | 1.02 | 0.04 | 1.01 | 0.04 | 1.07 | 0.02 | 0.99 | 0.02 | 1.06 | 0.04 |
| TSC2 | 0.92 | 0.01 | 0.95 | 0.06 | 0.94 | 0.07 | 0.96 | 0.03 | 1.01 | 0.07 | 0.93 | 0.03 |
| KLK3 | 1.02 | 0.11 | 0.95 | 0.08 | 1.04 | 0.14 | 1.01 | 0.14 | 0.98 | 0.06 | 0.99 | 0.09 |
| BRCA2 | 0.99 | 0.04 | 1.05 | 0.07 | 1.02 | 0.05 | 0.96 | 0.09 | 0.97 | 0.08 | 1.00 | 0.09 |
| TNFRSF7 | 0.94 | 0.06 | 1.02 | 0.11 | 0.89 | 0.11 | 1.02 | 0.15 | 1.04 | 0.12 | 0.94 | 0.08 |
| CASR | 0.96 | 0.11 | 0.9 | 0.1 | 0.86 | 0.05 | 0.81 | 0.13 | 1.00 | 0.16 | 0.96 | 0.11 |

Quantification of the methylation status of a particular CpG site was done by dividing the peak area with the combined areas of the control probes lacking a HhaI site. The relative peak area of each target probe from the digested sample was compared with those obtained from the undigested sample.
(—) indicates complete digestion of the MS-MLPA probe-DNA complex (absence of methylation).
Also depicted are the control probes without a HhaI site that were used for normalization and for the quantification of the methylation status. Methylation percentage is obtained by multiplying the average ratio by 100.

The P15^{INK4b} gene is commonly inactivated in association with promoter region hypermethylation involving multiple sites in its 5′-CpG Island (Chim, C. S., Liang, R., Tam, C. Y. and Kwong, Y. L. (2001) Methylation of p15 and p16 genes in acute promyelocytic leukemia: potential diagnostic and prognostic significance. J.Clin.Oncol., 19, 2033-2040). In some gliomas and all of the primary leukemia's, this event occurs without epigenetic alteration of the adjacent gene, p16^{INK4a}. In other tumors, including lung, head and neck, breast, prostate, and colon cancer, inactivation of p15^{INK4b} occurs only rarely and only with concomitant inactivation of p16^{(INK4a)} (Herman, J. G., supra; Herman, J. G., Civin, C. I., Issa, J. P., Collector, M. I., Sharkis, S. J. and Baylin, S. B. (1997) Distinct patterns of inactivation of p15INK4B and p16INK4A characterize the major types of hematological malignancies. Cancer Res., 57, 837-841; Dodge, J. E., List, A. F. and Futscher, B. W. (1998) Selective variegated methylation of the p15 CpG island in acute myeloid leukemia. Int.J.Cancer, 78, 561-567). Indeed, we did not observe hypermethylation of the p16^{INK4a} gene in any of the 21 AML samples.

To ensure that the disappearance of the MS-MLPA signals was not caused by any other event than the HhaI endonuclease treatment, we treated human genomic DNA (Promega) with HhaI methylase. In this way the internal cytosine residue in de HhaI recognition sequence (GCGC) becomes methylated. As expected, MS-MLPA with 20 ng of HhaI methylase treated DNA showed the presence of all MS-MLPA signals (data not shown), confirming that methylation of the sample DNA CpG sites prevents HhaI endonuclease digestion of the sample DNA probe hybrids.

Methylation Specific PCR (MSP) and Bisulphite Sequencing

To validate the MS-MLPA findings in the DNA samples from PWS and AS patients and DNA from AML samples, methylation-specific PCR (MSP) was carried out. The MSP primers were designed to amplify CpG regions in the SNRPN and p15 genes. Each primer pair was designed in order to contain at least two CpG sites including the ones that are recognized by MS-MLPA. As can be seen in FIG. 2d and 4, all samples that showed methylation of either SNRPN or p15 by MS-MLPA were also shown to be methylated by MSP. For the p15 gene we also performed bisulphite sequencing of the promoter region that is detected by the p15 MS-MLPA probe. Bisulphite sequencing was performed on three DNA samples: one control DNA sample treated with HhaI methylase (FIG. 5a), one DNA sample of an AML cell line that was negative for p15 methylation (FIG. 5b) and one AML cell line that was positive for p15 methylation after MS-MLPA (FIG. 5c).

The DNA sample that is treated with HhaI methylase only the internal cytosine residue of the GCGC sequence becomes methylated and thus is protected from bisulphite conversion which is clearly seen in FIG. 5a. All the other CpG-sites are converted (underlined). In the DNA sample negative for p15 methylation all the CpG site are converted (FIG. 5b), whereas in the sample with positive p15 methylation all the six CpG sites are protected including the CpG site (double underlined) recognized by the MS-MLPA probe (Fig. 5c).

MS-MLPA on Paraffin-Embedded Tissue

We next tested whether MS-MLPA could be used on DNA derived from formalin treated paraffin-embedded tissues. DNA extracted from paraffin material is usually of poor quality and is notoriously difficult to digest with restriction endonucleases. Storage of tissues in formaldehyde solution results in extensive crosslinking of proteins to other proteins and to nucleic acids and in nucleic acid fragmentation (Grunau, C., Clark, S. J. and Rosenthal, A. (2001) Bisulfite genomic sequencing: systematic investigation of critical experimental parameters. Nucleic Acids Res., 29, E65; Lehmann, U. and Kreipe, H. (2001) Real-time PCR analysis of DNA and RNA extracted from formalin-fixed and paraffin-embedded biopsies. Methods, 25, 409-418). Paraffin embedding is commonly used and results in partial denaturation of the DNA, making digestion of the sample DNA very difficult. In MS-MLPA, fragmentation of sample DNA is not a problem, since the probes only require 50-60 bp for hybridization and ligation. Besides, the sample DNA does not need to be double stranded as the digestion is performed on the MS-MLPA probe-DNA complex. Indeed, identical MS-MLPA results are obtained when using DNA derived from paraffin-embedded tissue as compared to fresh frozen material prepared from the same tumors (FIG. 6).

From the above experiments is can be shown that the claimed novel method, the MS-MLPA assay, is very suitable for detection of aberrant methylation patterns of CpG islands as well as copy number changes of a large number of genes in a simple reaction. To further validate this method and to show the linearity of response, genomic DNA samples of PWS and AS patients caused by uniparental disomy of chromosome 15 were analysed. The respective methylation status (0, 50 and 100% methylation) and copy number of the genes in the 2 Mb 15q11-q13 PWS/AS region could simply be identified by MS-MLPA. In addition, MS-MLPA was applied to DNA samples from AML cell lines. In line with previous reports, frequent aberrant promoter methylation of the tumor suppressor genes p15 and p73 were detected (Esteller, M., supra; Herman, J. G. et al., supra). For two genes, SNRPN and p15, the methylation status of the CpG sites recognized by the MS-MLPA probes were independently confirmed by MSP. To provide further evidence that the MLPA results are in agreement with the observed methylation status of this gene, bisulphite sequencing of the p15 promoter region was applied.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

What is claimed is:

1. Method for detecting in a sample, comprising a plurality of sample nucleic acids of different sequence, the presence of at least one methylated site on a specific location in a specific single stranded target nucleic acid sequence comprising a first and a second segment, and optionally a third segment being located between the first and second segments, the segments located essentially adjacent to one another, comprising, in a reaction mixture, the steps of:
1) incubating the sample nucleic acids with a plurality of different probe sets, allowing hybridisation of complementary nucleic acids to occur, each probe set comprising
a first nucleic acid probe having
a first target specific region complementary to the first segment of said target nucleic acid sequence and
a first non-complementary region, 3′ from the first region, being essentially non-complementary to said target nucleic acid sequence, comprising a first tag sequence,
a second nucleic acid probe having
a second target specific region complementary to the second segment of said target nucleic acid sequence and
a second non-complementary region, 5′ from the second region, being essentially non-complementary to said target nucleic acid sequence, comprising a second tag sequence,
and, optionally,
a third nucleic acid probe having
a third target specific region, complementary to the third segment,
2) connecting to one another the first, second and optionally the third probes, hybridised to the first, second and, if present, third segment of the same target nucleic acid sequence, respectively, the hybridised probes being located essentially adjacent to one another, forming a double-stranded connected probe assembly, wherein the sequence of at least one of the probes is chosen such that, in the connected probe assembly, a double-stranded recognition site for a methylation sensitive restriction enzyme is present,
3) incubating the connected probe assembly with the methylation sensitive restriction enzyme, allowing the methylation sensitive restriction enzyme to cleave the double-stranded connected probe assembly at unmethylated recognition sites, leaving methylated recognition sites intact,
4) amplifying the connected probe assembly, wherein amplification is initiated by binding of a first nucleic acid primer specific for the first tag sequence followed by elongation thereof,
5) detecting an amplicon.

2. Method according to claim 1, wherein at least steps 2) and 3) are performed simultaneously.

3. Method according to claim 1, wherein the amount of at least the first probe of at least one probe set in the mixture is less than 40 femtomoles, and the molar ratio between the first primer and the first probe being at least 200.

4. Method according to claim 3, wherein the amount of at least the first probe of each probe set in the mixture is less than 40 femtomoles, and the molar ratio between the first primer and the first probe being at least 200.

5. Method according to claim 3, wherein the molar ratio between the first primer and the first probe of at least one probe set is at least 400.

6. Method according to claim 5, wherein the molar ratio between the first primer and the first probe of at least one probe set is at least 800.

7. Method according to claim 6, wherein the molar ratio between the first primer and the first probe of at least one probe set is at least 1600.

8. Method according to claim 1, wherein the molar amount of at least the first probe of at least one probe set is less than 10 femtomoles.

9. Method according to claim 8, wherein the molar amount of at least the first probe of at least one probe set is less than 5 femtomoles.

10. Method according to claim 1, wherein at least 5 different probe sets are used of which the first tag sequences of the first nucleic acid probes are identical.

11. Method according to claim 1, wherein the amplification step comprises binding of a second nucleic acid primer, specific to the second tag sequence, to the elongation product of the first primer.

12. Method according to claim 1, wherein the molar amount of the second probe of at least one probe set is less than 40 femtomoles.

13. Method according to claim 12, wherein the molar amount of the second probe of at least one probe set is less than 10 femtomoles

14. Method according to claim 13, wherein the molar amount of the second probe of at least one probe set is less than 5 femtomoles.

15. Method according to claim 1, wherein the molar ratio between the second primer and the second probe is at least 200.

16. Method according to claim 15, wherein the molar ratio between the second primer and the second probe is at least 500.

17. Method according to claim 16, wherein the molar ratio between the second primer and the second probe is at least 1000.

18. Method according to claim 17, wherein the molar ratio between the second primer and the second probe is at least 2000.

19. Method according to claim 1, wherein at least 5 different probe sets are used of which the second tag sequences of the second nucleic acid probes are identical.

20. Method according to claim 1, wherein the molar ratio between the second primer and the total amount of probes present in the reaction mixture is at least 5.

21. Method according to claim 20, wherein the molar ratio between the second primer and the total amount of probes present in the reaction mixture is at least 15.

22. Method according to claim 21, wherein the molar ratio between the second primer and the total amount of probes present in the reaction mixture is at least 25.

23. Method according to claim 1, wherein the reaction mixture comprises at least 10 different sets of probes.

24. Method according to claim 23, wherein the reaction mixture comprises at least 20 different sets of probes.

25. Method according to claim 24, wherein the reaction mixture comprises 30-60 different sets of probes.

26. Method according to claim 1, wherein at least a portion of the unhybridised probes remains in the reaction mixture at least during steps 1-4.

27. Method according to claim 1, wherein all unhybridised probes remain in the reaction mixture during at least steps 1-4.

28. Method according to claim 1, wherein steps 1-4 are carried out in the same reaction vessel, the reaction mixture not being removed from the said vessel during said steps.

29. Method according to claim 1, wherein, in a reaction mixture of 3-150 μl, the amount of:
sample nucleic acid is 10-1000 ng,
the first probe of each probe set is 0.5-40 fmol,
the second probe of each probe set is 0-40 fmol,
each first primer is 5-20 pmol,
each second primer is 0-20 pmol.

30. Method according to claim 1, wherein the reaction mixture, at least during step 2), comprises ligation activity, connecting the essentially adjacent probes.

31. Method according to claim 30, wherein at least during step 3), any ligation activity present is incapable of ligating double-stranded nucleic acids.

32. Method according to claim 30, wherein the ligation activity is obtained by providing a thermostable nucleic acid ligase, at least 95% of the activity being inactivated within ten minutes above a temperature of approximately 95° C.

33. Method according to claim 1, wherein at least one nucleic acid probe comprises an enzymatic template directed polymerised nucleic acid.

34. Method according to claim 33, wherein at least one probe is generated by digestion of DNA with a restriction endonuclease.

35. Method according to claim 34, wherein the probe generating restriction endonuclease is capable of cutting at least one strand of the DNA outside the enzyme recognition site sequence on said DNA.

36. Method according to claim 34, wherein the DNA used is single stranded DNA made partially double stranded by annealing of one or more oligonucleotides.

37. Method according to claim 1, wherein at least one probe comprises two separate probe parts being connected together in step 2).

38. Method according to claim 37, wherein at least one of said probe parts comprises enzymatic template directed polymerised nucleic acid.

39. Method according to claim 1, further comprising extending a 3′ end of a hybridised probe prior to step 2).

40. Method according to claim 1, further comprising providing said sample with a competitor nucleic acid comprising a nucleic acid sequence capable of competing with at least one probe for hybridisation to a target nucleic acid.

41. Method according to claim 1, wherein said sample is further provided with a known amount of a target sequence for one or more probe pairs, prior to step 2).

42. Method according to claim 1, wherein said sample is further provided with a known amount of one or more connected probes, prior to step 4).

43. Method according to claim 1, further comprising quantification of the relative or absolute abundance of a target nucleic acid in said sample, wherein from a part of the reaction mixture, step 4) is omitted.

44. Method according to claim 43, for determining the absolute or relative abundance of multiple single stranded target nucleic acids in the sample.

45. Method according to claim 1 for detecting a nucleotide polymorphism, preferably a single nucleotide polymorphism.

46. Method according to claim 1, for the detection of multiple methylated single stranded target nucleic acids.

47. Method according to claim 46, wherein said multiple methylated single stranded target nucleic acids are detected through the detection of multiple amplicons.

48. Method according to claim 47, wherein at least two of said multiple amplicons can be discriminated on the basis of a difference in size of said at least two amplicons.

49. Method according to claim 48, wherein the methylated site comprises a cytosine nucleotide adjacently located 5′ to a guanine nucleotide.

50. Nucleic acid probe for use in a method according to claim 1, comprising a single stranded sequence, constituting one of the strands of the double stranded recognition site of the methylation sensitive restriction enzyme.

51. Nucleic acid probe for use in a method according to claim 1, comprising at least at one of the termini thereof, at least a part of a single stranded sequence, constituting one of the strands of a double stranded recognition site of the methylation sensitive restriction enzyme.

52. Nucleic acid probe for use in a method according to claim 33, comprising a single stranded sequence, constituting one of the strands of a double stranded recognition site of the methylation sensitive restriction enzyme.

53. Nucleic acid probe for use in a method according to claim 33, comprising at least at one of the termini thereof, at least a part of a single stranded sequence, constituting one of the strands of a double stranded recognition site of the methylation sensitive restriction enzyme.

54. Mixture of at least a first nucleic acid probe and a second nucleic acid probe and optionally a third nucleic acid probe as defined in claim 1, wherein at least one of the probes comprises a single stranded sequence, constituting one of the strands of a double stranded recognition site of the methylation sensitive restriction enzyme.

55. Mixture of at least a first nucleic acid probe and a second nucleic acid probe and optionally a third nucleic acid probe as defined in claim 1, wherein one of the strands of a double stranded recognition site of the methylation sensitive restriction enzyme is formed when the 3′ end of the first probe is connected to the 5′ end of the second probe, or, if the third probe is present, the 3′ end of the first probe is connected to the 5′ end of the third probe or the 3′ end of the third probe is connected to the 5′ end of the second probe.

56. Mixture of at least a first nucleic acid probe and a second nucleic acid probe and optionally a third nucleic acid probe as defined in claim 33, wherein at least one of the probes comprises a single stranded sequence, constituting one of the strands of a double stranded recognition site of the methylation sensitive restriction enzyme.

57. Mixture of at least a first nucleic acid probe and a second nucleic acid probe and optionally a third nucleic acid probe as defined in claim 33, wherein one of the strands of a double stranded recognition site of the methylation sensitive restriction enzyme is formed when the 3′ end of the first probe is connected to the 5′ end of the second probe, or, if the third probe is present, the 3′ end of the first probe is connected to the 5′ end of the third probe or the 3′ end of the third probe is connected to the 5′ end of the second probe.

58. Kit for performing the method according to claim 1, comprising a nucleic acid probe for use in the method according to claim 1, said nucleic acid probe comprising a single stranded sequence, constituting one of the strands of the double stranded recognition site of the methylation sensitive restriction enzyme.

59. Kit for performing the method according to claim 1, comprising a nucleic acid probe for use in the method according to claim 1, said nucleic acid probe comprising at least at one of the termini thereof, at least a part of a single stranded sequence, constituting one of the strands of a double stranded recognition site of the methylation sensitive restriction enzyme.

60. Kit for performing the method according to claim 33, comprising a nucleic acid probe for use in the method according to claim 33, said nucleic acid probe comprising a single stranded sequence, constituting one of the strands of a double stranded recognition site of the methylation sensitive restriction enzyme.

61. Kit for performing the method according to claim 33, comprising a nucleic acid probe for use in the method according to claim 33, said nucleic probe comprising at least at one of the termini thereof, at least a part of a single stranded sequence, constituting one of the strands of a double stranded recognition site of the methylation sensitive restriction enzyme.

62. Kit for performing the method according to claim 1, comprising a mixture of nucleic acid probes which comprises at least a first nucleic acid probe and a second nucleic acid probe and optionally a third nucleic acid probe as defined in claim 1, wherein at least one of the probes comprises a single stranded sequence, constituting one of the strands of a double stranded recognition site of the methylation sensitive restriction enzyme.

63. Kit for performing the method according to claim 1, comprising a mixture of nucleic acid probes which comprises at least a first nucleic acid probe and a second nucleic acid probe and optionally a third nucleic acid probe as defined in claim 1, wherein one of the strands of a double stranded recognition site of the methylation sensitive restriction enzyme is formed when the 3′ end of the first probe is connected to the Slend of the second probe, or, if the third probe is present, the 3′ end of the first probe is connected to the 5′ end of the third probe or the 3′ end of the third probe is connected to the 5′ end of the second probe.

64. Kit for performing the method according to claim 33, comprising a mixture of nucleic acid probes which comprises at least a first nucleic acid probe and a second nucleic acid probe and optionally a third nucleic acid probe as defined in claim 33, wherein at least one of the probes comprises a single stranded sequence, constituting one of the strands of a double stranded recognition site of the methylation sensitive restriction enzyme.

65. Kit for performing the method according to claim 33, comprising a mixture of nucleic acid probes which comprises at least a first nucleic acid probe and a second nucleic acid probe and optionally a third nucleic acid probe as defined in claim 33, wherein one of the strands of a double stranded recognition site of the methylation sensitive restriction enzyme is formed when the 3′ end of the first probe is connected to the 5′ end of the second probe, or, if the third probe is present, the 3′ end of the first probe is connected to the 5′ end of the third probe or the 3′ end of the third probe is connected to the 5′ end of the second probe.
